SEQUENCE LISTING (55 sequences)

SEQ ID NO: 1 - DNA, 20 bp, Artificial Sequence
  Forward primer
gccagaagtgcaggagttgg

SEQ ID NO: 2 - DNA, 20 bp, Artificial Sequence
  Forward primer
ctgttggtgatgatggcact

SEQ ID NO: 3 - DNA, 18 bp, Artificial Sequence
  Forward primer
actgggaatgcaggtgga

SEQ ID NO: 4 - DNA, 20 bp, Artificial Sequence
  Forward primer
cactgagtggtccaaatgca

SEQ ID NO: 5 - DNA, 20 bp, Artificial Sequence
  Forward primer
agacaactgggaatgcaggt

SEQ ID NO: 6 - DNA, 19 bp, Artificial Sequence
  Forward primer
caagcacacctgctctgca

SEQ ID NO: 7 - DNA, 24 bp, Artificial Sequence
  Forward primer
ggtgaagtttatgacaaagaaagc

SEQ ID NO: 8 - DNA, 18 bp, Artificial Sequence
  Forward primer
tggtgtcatgggatgcag

SEQ ID NO: 9 - DNA, 21 bp, Artificial Sequence
  Forward primer
cacatcaacttccatggagaa

SEQ ID NO: 10 - DNA, 20 bp, Artificial Sequence
  Forward primer
gcagaggcattcgtgaatct

SEQ ID NO: 11 - DNA, 28 bp, Artificial Sequence
  Reverse primer
cagtgaacagagtaaaacaaacacagag

SEQ ID NO: 12 - DNA, 19 bp, Artificial Sequence
  Reverse primer
caatggctgaacatggtgc

SEQ ID NO: 13 - DNA, 18 bp, Artificial Sequence
  Reverse primer
actatggcggtgctgtgg

SEQ ID NO: 14 - DNA, 29 bp, Artificial Sequence
  Reverse primer
ggctttcatatttttattcagaatctatc

SEQ ID NO: 15 - DNA, 29 bp, Artificial Sequence
  Reverse primer
cctgatagtattactttacacactgagaa

SEQ ID NO: 16 - DNA, 27 bp, Artificial Sequence
  Reverse primer
cacatgggaaaattaacattaacatta

SEQ ID NO: 17 - DNA, 25 bp, Artificial Sequence
  Reverse primer
cagtttcgaacaagtatattggatc

SEQ ID NO: 18 - DNA, 20 bp, Artificial Sequence
  Reverse primer
gcacaagtgtgttggacgag

SEQ ID NO: 19 - DNA, 26 bp, Artificial Sequence
  Reverse primer
tttccatagtttcgatacaccagtaa

SEQ ID NO: 20 - DNA, 20 bp, Artificial Sequence
  Reverse primer
taacatccaacgaaccacgg

SEQ ID NO: 21 - DNA, 21 bp, Artificial Sequence
  Reverse primer
aagacaacgaaggacgtgaca

SEQ ID NO: 22 - DNA, 20 bp, Artificial Sequence
  Reverse primer
ccgatgcctggatattcatg

SEQ ID NO: 23 - DNA, 22 bp, Artificial Sequence
  Reverse primer
ccaacatgactgcatttctacc

SEQ ID NO: 24 - protein, 152 aa, Populus trichocarpa
MVKAVAVLNSSEGVSGTIFFTQEGDGPTTVTGNLSGLKPGLHGFHVHALGDTTNGCMSTGPHFNPVGKEHGAPEDENRHAGDLGNVTVGDDGTATFTIIDKQIPLTGPHSIIGRAVVVHGDPDDLGKGGHELSKTTGNAGGRVACGIIGLQG

SEQ ID NO: 25 - protein, 152 aa, Populus trichocarpa
MVKAVAVLNSSEGVKGTINFTQEGDGPTTVTGSLCGLKPGLHGFHVHALGDTTNGCMSTGPHFNPVGKEHGAPEDENRHAGDLGNVTVGDDGTATVSIIDNQIPLTGPNSIVGRAVVVHADPDDLGKGGHELSKTTGNAGGRVACGVIGLQG

SEQ ID NO: 26 - protein, 152 aa, Arabidopsis thaliana
MAKGVAVLNSSEGVTGTIFFTQEGDGVTTVSGTVSGLKPGLHGFHVHALGDTTNGCMSTGPHFNPDGKTHGAPEDANRHAGDLGNITVGDDGTATFTITDCQIPLTGPNSIVGRAVVVHADPDDLGKGGHELSLATGNAGGRVACGIIGLQG

SEQ ID NO: 27 - protein, 210 aa, Populus trichocarpa
MAAHAILTATPPTRYPLLSPLPPNHSSFHGVSLKLPRQSFSFSLAAKKQQPPFVVAATKKAVAVLKGTSSVEGVVTLSQEDDGPTTVSVRITGLTPGPHGFHLHEFGDTTNGCMSTGPHFNPKKLTHGAPEDEIRHAGDLGNIVANADGVAEATIVDSQIPLSGPNAVIGRALVVHELEDDLGKGGHELSSTTGNAGGRLACGVVGLTPI

SEQ ID NO: 28 - protein, 210 aa, Populus trichocarpa
MAAHAILTATPPTRHPLLSPLPPNHSSFHGVSLKLPRQSFSFSLAAKKQQPPFVVAATKKAVAVLKGTSSVEGVVTLSQEDDGPTTVSVRITGLTPGPHGFHLHEFGDTTNGCMSTGAHFNPKKLTHGAPEDEIRHAGDLGNIVANADGVAEATIVDSQIPLSGPNAVIGRALVVHELEDDLGKGGHELSSTTGNAGGRLACGVVGLTPI

SEQ ID NO: 29 - protein, 219 aa, Populus trichocarpa
MQAAAIAAMAAHAILTAMPPILHLPLSPLPPNHSSFHGVSLNPPRQSFSLSLAAKKQPPLFVVASATKKAVAVLKGTSNVEGVVILTQEADGPTTVNARITGLTPGPHGFHLHQYGDTTNGCVSTGAHFNPNNLTHGAPEDEIRHAGDLGNIVATADGVAEAIIVDNQIPLSGPNTVIGRALVVHELEDDLGKGKHELSSTTGNAGGRLACGVVGLTPV

SEQ ID NO: 30 - protein, 216 aa, Arabidopsis thaliana
MAATNTILAFSSPSRLLIPPSSNPSTLRSSFRGVSLNNNNLHRLQSVSFAVKAPSKALTVVSAAKKAVAVLKGTSDVEGVVTLTQDDSGPTTVNVRITGLTPGPHGFHLHEFGDTTNGCISTGPHFNPNNMTHGAPEDECRHAGDLGNINANADGVAETTIVDNQIPLTGPNSVVGRAFVVHELKDDLGKGGHELSLTTGNAGGRLACGVIGLTPL

SEQ ID NO: 31 - protein, 158 aa, Populus trichocarpa
MATGSVKAVALITGDSNVRGSLHFIQEPNGATHVTGRIAGLSPGLHGFHIHALGDTTNGCNSTGPHFNPLKKDHGAPCDNERHAGDLGNIIAGSDGVAEVSITDFQIPLSGMHSILGRAVVVHADPDDLGKGGHDLSKTTGNAGARVGCGIIGLKSSV

SEQ ID NO: 32 - protein, 158 aa, Populus trichocarpa
MATGSVKAVALITGDSIVRGSLHFIQEPNGATHVTGRITGLSPGLHGFHIHALGDTTNGCNSTGPHFNPLKKDHGAPSDKERHAGDLGNIIAGSDGVAEVSIKDLQIPLSGMHSILGRAVVVHADPDDLGKGGHELSKTTGNAGARVGCGIVGLKSSV

SEQ ID NO: 33 - protein, 164 aa, Arabidopsis thaliana
MEAPRGNLRAVALIAGDNNVRGCLQFVQDISGTTHVTGKISGLSPGFHGFHIHSFGDTTNGCISTGPHFNPLNRVHGPPNEEERHAGDLGNILAGSNGVAEILIKDKHIPLSGQYSILGRAVVVHADPDDLGKGGHKLSKSTGNAGSRVGCGIIGLQSSADAKL

SEQ ID NO: 34 - protein, 323 aa, Populus trichocarpa
MAFLRSVTNTTKVAIPASALPDASALSSISTSSPYPKPRNLSFLSPSSSLLSSTASGLVKNLTQPPSALNMETPASNHMSISQDNVTLPELFTEFMVDMKCDGCVNSVKNKLQSVNGVKNVEVDLANQVVRILGSSPVNTMTEALEQTGRNARLIGQGIPEDFLVSAAVAEFKGPEIFGVVRFAQVNMDLARIEANFTGLSLGKHGWSINEFGDLTEGAASTGKVFNPTNQGTELEPLGDLGTLYVDEKGDAFFSGTIQKLRIADLIGRSLVVYGTEDKSDKGLTAAVIARSAGVGENYKKICTCDGTTIWESSNNDFVAGKV

SEQ ID NO: 35 - protein, 323 aa, Populus trichocarpa
MAFLRSVTTTTKTAIAALALPAAFAFSSLSPSSPNPKPENLFFLSPSSSLLTSARFGLVKNLTQRPSSLSMDTSTSNQKPISQDNGALPEILTEFMVDMKCEGCVNSVRNKLQAVNGVKNVEVDLANQVVRILGSSPVKTMTEALEQTGRNARLIGQGIPEDFLVSAAVAEFKGPDIFGVVRFAQVNMELARIEASFSGVSPGKHGWSINEFGDLTKGAASTGKVFNPTNQGTEQEPLGDLGTLDVDEKGDAFFSGTKRKLRVADLIGRSVVLFGTEDKSDEGLTAAVIARSAGVGENYKKICTCDGTIIWESSNNDFVASKV

SEQ ID NO: 36 - protein, 320 aa, Arabidopsis thaliana
MASILRSVATTSAVVAAASAIPIAIAFSSSSSSSSTNPKSQSLNFSFLSRSSPRLLGLSRSFVSSPMATALTSDRNLHQEDRAMPQLLTEFMVDMTCEGCVNAVKNKLETIEGIEKVEVDLSNQVVRILGSSPVKAMTQALEQTGRKARLIGQGVPQDFLVSAAVAEFKGPDIFGVVRFAQVSMELARIEANFTGLSPGTHSWCINEYGDLTNGAASTGSLYNPFQDQTGTEPLGDLGTLEADKNGEAFYSGKKEKLKVADLIGRAVVVYKTDDNKSGPGLTAAVIARSAGVGENYKKLCSCDGTVIWEATNSDFVASKV

SEQ ID NO: 37 - protein, 129 aa, Artificial Sequence
  Consensus
IGSKAKAVAVLKGSVGVVFTQEGTTVTGRITGLTPGHGFHVHFGDTTNGCMSTGPHFNPHGAPEDERHAGDLGNIADGVAETIDQIPLTGPSIIGRAVVVHGDDLGKGGHELSTTGNAGGRVACGIIGL

SEQ ID NO: 38 - protein, 229 aa, Populus trichocarpa
MALRSLVCRKSLGLGSSSSFKLHFRGLQTFSLPDLPYDYGALEPAISGEIMQLHHQKHHQTYITNYNKAVEQLHHAMEKGDSSAVVKLQSAIKFNGGGHVNHSIFWKNLSPVSERGGESPHGSLGWAIDEHFGSLDALIQKMSTEGAALQGSGWVWFGLDKESKKLVVETTANQDPLVTKGPLVPLLGIDVWEHAYYLQYKNARPDYLKNIWKVMNWKYAGEVYDKEHS

SEQ ID NO: 39 - protein, 225 aa, Populus trichocarpa
MALRSLVSRKTLGLGLKLQFRGLQTFSLPDLPYDYGALEPAISGEIMQLHHQKHHQTYITNYNKSLEQLHHAMEKGDSSAVVKLQSAIKFNGGGHVNHSIFWKNLTPVQEGGGEPPHGRLGWAIDEDFGSLESLIKKMSTEGAAVQGSGWVWLGLDKESKKLVVETTENQDPLVTKGPLVPLLGVDVWEHAYYLQYKNVRPDYLKNIWKVMNWKYAGEVYDKESS

SEQ ID NO: 40 - protein, 231 aa, Arabidopsis thaliana
MAIRCVASRKTLAGLKETSSRLLRIRGIQTFTLPDLPYDYGALEPAISGEIMQIHHQKHHQAYVTNYNNALEQLDQAVNKGDASTVVKLQSAIKFNGGGHVNHSIFWKNLAPSSEGGGEPPKGSLGSAIDAHFGSLEGLVKKMSAEGAAVQGSGWVWLGLDKELKKLVVDTTANQDPLVTKGGSLVPLVGIDVWEHAYYLQYKNVRPEYLKNVWKVINWKYASEVYEKENN

SEQ ID NO: 41 - protein, 307 aa, Populus trichocarpa
MVAAAATATAANPLTCSLFPSQGLRGTTSGLQWTKRQCTRKASALTVTAKFELKPPPYPMNALEPHMSKDTFEYHWGKHHRAYVNNLNKQIEGTELDDMSLDDVVLITYNKGDPLPAFNNAAQAWNHEFFWESMKPGGGGKPSGELLQLMERDFGSFDKFVEEFKSAAATQFGSGWAWLVYKANRLDVGNAVNPCPSEEDKKLVVVKSPNAVNPLVWDYHPLLTIDVWEHAYYLDFQNRRPDYTSNFMERLVSWDAVSARLEVARARAAEREIEEARKRKEEEEGNFTDKEPAEIFIDGVADDSETD

SEQ ID NO: 42 - protein, 305 aa, Arabidopsis thaliana
MMNVAVTATPSSLLYSPLLLPSQGPNRRMQWKRNGKRRLGTKVAVSGVITAGFELKPPPYPLDALEPHMSRETLDYHWGKHHKTYVENLNKQILGTDLDALSLEEVVLLSYNKGNMLPAFNNAAQAWNHEFFWESIQPGGGGKPTGELLRLIERDFGSFEEFLERFKSAAASNFGSGWTWLAYKANRLDVANAVNPLPKEEDKKLVIVKTPNAVNPLVWDYSPLLTIDTWEHAYYLDFENRRAEYINTFMEKLVSWETVSTRLESAIARAVQREQEGTETEDEENPDDEVPEVYLDSDIDVSEVD

SEQ ID NO: 43 - protein, 212 aa, Arabidopsis thaliana
MAASSAVTANYVLKPPPFALDALEPHMSKQTLEFHWGKHHRAYVDNLKKQVLGTELEGKPLEHIIHSTYNNGDLLPAFNNAAQAWNHEFFWESMKPGGGGKPSGELLALLERDFTSYEKFYEEFNAAAATQFGAGWAWLAYSNEKLKVVKTPNAVNPLVLGSFPLLTIDVWEHAYYLDFQNRRPDYIKTFMTNLVSWEAVSARLEAAKAASA

SEQ ID NO: 44 - protein, 263 aa, Arabidopsis thaliana
MSSCVVTTSCFYTISDSSIRLKSPKLLNLSNQQRRRSLRSRGGLKVEAYYGLKTPPYPLDALEPYMSRRTLEVHWGKHHRGYVDNLNKQLGKDDRLYGYTMEELIKATYNNGNPLPEFNNAAQVYNHDFFWESMQPGGGDTPQKGVLEQIDKDFGSFTNFREKFTNAALTQFGSGWVWLVLKREERRLEVVKTSNAINPLVWDDIPIICVDVWEHSYYLDYKNDRAKYINTFLNHLVSWNAAMSRMARAEAFVNLGEPNIPIA

SEQ ID NO: 45 - protein, 308 aa, Populus trichocarpa
MKSFTQNQFNPTLPSLYQVKNCSKITGQERVDCTTKMGCFSYNLFSSSPSCQVPVTGSFACQFKTTPYKQLPTLFKQKKKHFNGCQRASRVVSYYALKTPPYKLDALEPYMSKRTVEVHWKEHHGAYVEGLNKALAKSDILYGYTLDDLVKVTYNNGNPSPDFNNAAQVWNHDFFWESMQPGGGDMPELGMLEQIEKDFGSFTNFRNKFAETALTLFGSGWVWLVLKREEPRLEVVKTLNAVTPIVWGDIPIINLDMWEHAYYLDYKNDKENYVNAFMDHLVSWNMAMARMARAEAFVNLGEPKIPIA

SEQ ID NO: 46 - protein, 192 aa, Artificial Sequence
  Consensus
LSGLRMKSGRRTVRVVAFLKPPYLDALEPMSKETLEVHWGKHHRAYVDNLNKQLETDLGSLEIISTYNKGNLPFNNAAQVWNHEFFWESMPGGGDPGLLIERDFGSFEFVEKFTSAAATFGSGWVWLALKREEKLVVVKTSNAVNPLVWIPLLIDVWEHAYYLDYKNRPDYIKNFMRLVSWASRLERAAEII

SEQ ID NO: 47 - DNA, 66 bp, Populus trichocarpa
atgtttttcttaccttcactctcttttttctttcttcatccgactcagtgttttcttgcatttcag

SEQ ID NO: 48 - DNA, 39 bp, Populus trichocarpa
ggttgccagtattacacgttcggtcagtcagaacttttg

SEQ ID NO: 49 - DNA, 75 bp, Populus trichocarpa
aggcaatgtaccagaaaggctgatgctttgacagttacagctaaatttgagctgaaacctcctccatatcccatg

SEQ ID NO: 50 - DNA, 189 bp, Populus trichocarpa
gatgatttagagccgcatatgagcaaggacacatttgagtatcaccggggaaagcatcacagggcttatgtggataacttaaacaagcaaattgacggaacagaacgagatgacatgtccttagatgatgttgtgctcgttacatacaacaagggtggtccacttcctgctttcaacaatgctgcacag

SEQ ID NO: 51 - DNA, 172 bp, Populus trichocarpa
gcatggaaccatgaattctttttggaatccatgaaaccgggaggtggaggaaaggcatcaggggaacttcttcacttgattgaaagagattttggttcttttgatagatttgtgcaagagttcaagtcggctgcagctactcagtttggttctggatgggcttggcttgttt

SEQ ID NO: 52 - DNA, 106 bp, Populus trichocarpa
acaaggaaaatagacttagatgctggaaatgttgtaaatcctcgtccaccggaggaggataaaaagcttgtagtggtcaagagtcccagtgcagtaaagccccctt

SEQ ID NO: 53 - DNA, 27 bp, Populus trichocarpa
ccactccatacaattgatgcttgggag

SEQ ID NO: 54 - DNA, 24 bp, Populus trichocarpa
tatgcatactacctggactttcag

SEQ ID NO: 55 - DNA, 191 bp, Populus trichocarpa
aatcgacgacctgattacacatcaacttccatggagaagcttgtgtttagtgcgagacttgaagttgcaaaggctcgagctgccgagagagaaatagaagaagcgagaaagaaaaaggaggaactttatagacaaaaaaacctgccgaggttttcgtggatgggatagctgatgattctgagactgattaa

(19) United States
(12) Patent Application Publication
(10) Pub. No.: US 2015/0067914 A1
(43) Pub. Date: Mar. 5, 2015

(54) Drought Stress Tolerance Genes and Methods of Use Thereof to Modulate Drought Resistance in Plants

(71) Applicants: Edward G. Kirby, Basking Ridge, NJ (US); Juan Jesus Molina-Rueda, Malaga (ES)

(72) Inventors: Edward G. Kirby, Basking Ridge, NJ (US); Juan Jesus Molina-Rueda, Malaga (ES)

(21) Appl. No.: 14/314,934

(22) Filed: Jun. 25, 2014

Related U.S. Application Data
(60) Provisional application No. 61/839,124, filed on Jun. 25, 2013.

(51) Int. Cl. C12N 15/82; C12Q 1/68

(52) U.S. Cl. CPC C12N 15/8273; C12Q 1/6895; C12Q 2600/13; C12Q 2600/16
USPC 800/278; 435/320.1; 800/298; 435/419; 506/17

(57) ABSTRACT

The present invention has increased the resistance to drought stress in Poplar by integrating a transgene constitutively expressing a pine superoxide dismutase (SOD) into the plant genome. It is contemplated that this approach to drought resistance improvement will be equally successful for all woody perennials. Provided with the invention is an expression cassette, a vector, and a method for increasing SOD activity in woody perennials, as well as transgenic woody perennials with enhanced drought resistance and accompanying phenotype.

This application claims priority to U.S. Provisional Patent Application No. 61/839,124 filed Jun. 25, 2014, the entire contents being incorporated herein by reference as though set forth in full.

Pursuant to 35 U.S.C. §202(c) it is acknowledged that the U.S. Government has rights in the invention described, which was made with funds from the US Department of Energy, Grant No. GO12026-314.

### FIELD OF THE INVENTION

This invention relates to the field of plant breeding, forestry, plant transformation, and mineral nutrition. More specifically, a transgenic woody perennial plant is provided, having improved responses drought stress.

### BACKGROUND OF THE INVENTION

Several publications and patent documents are cited throughout the specification in order to describe the state of the art to which this invention pertains. Each of these references are incorporated herein as though set forth in full.

Inorganic nitrogen (N) is the most limiting nutrient affecting the growth of forest trees. As N uptake is influenced by soil water availability [1,2], this problem is exacerbated by increasingly frequent episodes of drought in many regions of the world due to ongoing climate change [3]. In addition to the adverse effects on mineral nutrient uptake, drought causes oxidative stress in plants, including poplar [4,5]. As such, the drought stress response is tightly coupled with the antioxidant defense system and cellular redox regulation [6].

Glutamine synthetase (GS) plays a central role in assimilation of ammonium into amino acids and other reduced N compounds in plants. Consistent with the central importance of N metabolism in plant growth and development, hybrid poplar (Populus tremula×alba, INRA 717-1B4) expressing ectopically the pine glutamine synthetase gene (GS1a) exhibited several pleiotropic phenotypes of agronomic significance. These include increased growth [7,8], increased nitrogen use efficiency [9], altered wood chemistry [10], and of particular relevance to the present investigation, enhanced tolerance to drought [11].

The superoxide dismutases (SODs) constitute a first line of defense against reactive oxygen species (ROS) [12]. SODs are metalloenzymes that catalyze the dismutation of ion superoxide into oxygen and hydrogen peroxide [13]. The superoxide radical is a ROS whose production increases under abiotic and biotic stresses, including drought [14]. Thus, SODs play a critical role in protecting plant tissues from ROS [12]. SODs are classified according to their metal cofactors and/or subcellular distribution. The predominant forms of SOD in plants are mitochondrial manganese SODs (MnSODs), cytosolic copper/zinc SODs (Cu/ZnSODs), chloroplastic Cu/ZnSODs, and iron SODs (FeSODs) [15]. In addition, plant SODs have been localized in peroxisomes, glyoxysomes [16], vacuoles, the nucleus [17], and the extracellular matrix [18]. Expression of plant SOD genes is regulated by developmental and environmental cues, including hormones [19,20], high light and UV [15], and drought [21]. Recent work at the molecular level has shown that SOD expression can be modulated by alternative splicing [18,22] and microRNAs [23,24]. Transgenic plants that over-express SOD genes display a range of phenotypes depending on the targeted SOD isoform, the level of transgene expression, and subcellular distribution.

### SUMMARY OF THE INVENTION

The present invention relates to the production of transgenic woody perennial plants having improved drought resistance due to expression of chimeric transgenes, comprising the coding sequence of at least one superoxide dismutate (SOD) gene selected from the group consisting of PtFSD2.1 and PtFSD3 or the putative iron transporter PtYSL (which is putatively involved in providing iron for the iron SODs) operably linked to appropriate 5′ and 3′ regulatory sequences. In an alternative embodiment the plants express glutamine synthetase and at least one SOD gene. The present invention particularly relates to altering the expression of SOD enzymes in such plants, thereby improving numerous agronomic, economical and environmental features of the plants, such as their ability to grow under stress conditions. Other improvements found in these transgenic plants can include enhanced or novel phenotypes, such as faster growth, higher biomass production, and higher nutritional quality of fruit, seeds and foliage.

One aspect of the invention is a plant expression cassette that will alter the level and location of SOD in plants. This expression cassette comprises a SOD gene operably linked to a promoter. In preferred embodiments, the SOD gene is from a gymnosperm, the genus Pinus, and the species Pinus sylvestris. In other preferred embodiments, the expression cassette additionally comprises the cauliflower mosaic virus 35S promoter and the NOS terminator.

Another aspect of the invention is a vector containing the expression cassette. In preferred embodiments, the vector is an Agrobacterium binary vector and pBIN19. In another preferred embodiment, the vector comprises the neomycin phosphotransferase II coding sequence.

Another aspect of the invention is a method for producing a transgenic plant with enhanced drought resistance by transforming a plant in vitro with the aforementioned expression cassette. In preferred embodiments, the plant is a woody perennial, in the family Salicaceae, in the genus Populus, a hybrid Populus tremula×P. alba, and clone INRA 717 1-B4 of the hybrid Populus tremula×P. alba. In other preferred embodiments, the method uses Agrobacterium tumefaciens and the Agrobacterium binary vector containing the glutamine synthetase expression cassette. This aspect includes a transgenic plant made by the method and a reproductive unit from the plant.

Another aspect of the invention is a transgenic woody perennial plant with improved stress resistance which comprises at least one transgene expressing the coding sequence of a SOD.

In yet another aspect of the invention a panel of isolated drought resistance nucleic acid biomarkers optionally affixed to a solid support are provided. In a one aspect of the invention, these isolated nucleic acids are provided in Table 1.

### BRIEF DESCRIPTION OF THE DRAWINGS

FIG. 1. Alignment of predicted SOD and CCS amino acid sequences from Populus trichocarpa and Arabidopsis thaliana. Blue boxes in the amino termini and underlined sequences in carboxy termini represent predicted transit peptides (see Table 1 for details). Alignments were generated using ClustalX 2.0.12 [30]. Boxes showing identical (black) and similar (grey) amino acids and the consensus sequence were included in the alignment by Boxshade 3.21 (www.ch.embnet.org/software/BOX_form.html). A. CSD and CCS alignment. Amino acids involved in copper binding for CCSs in the consensus region MXCXXC [51] are marked with pluses (+) in their amino termini. Amino acids involved in metal binding for CSD group [52] are marked with asterisks (*). Sequences are SEQ ID NOs: 24-37, from top to bottom. B. MSD and FSD alignment. Metal ligands [53] and the tyrosine residue essential for catalytic activity [22] are marked with asterisks (*) and tail arrows (↓), respectively. The primary candidates for distinguishing MSD from FSD [72] are indicated with solid arrowheads. Tryptophan residues within this region may confer H₂O₂ sensitivity in FSDs [73]. Sequences are SEQ ID NOs: 38-46, from top to bottom. C. Expression of poplar reference genes selected for RT-qPCR analysis across all tissues and conditions in the present study. The three reference genes were selected according to Vandesompele et al. [40] and validated as reference genes: elongation factor 1β (EF1β), actin (ACT), and ubiquitin (UBQ). Samples for sets 1 and 2 are ordered as follows: sink leaf, source leaf, stem, main root and fine roots in well-watered, drought and recovery. Values for pairwise variation for the three reference genes (V3) considering their expression in all samples, were calculated using geNorm. V3 values obtained (0.098 and 0.13 for the first and second replicates, respectively) were lower than the cut-off (0.15) proposed by Vandesompele et al. [40]. Values are presented as quantitative cycles (Cq) for each of the three reference genes.

FIG. 2. Phylogenetic analysis of Populus trichocarpa (Pt) and Arabidopsis thaliana (At) SODs based on predicted amino acid sequences. The neighbor-joining tree was generated using Mega 5.05 [31]. The bootstrap method with 1000 replicates was used as a test of the phylogeny. The three groups identified include the copper/zinc SODs (triangles), manganese SODs (squares), and iron SODs (circles). Poplar and Arabidopsis sequences are marked with solid and empty symbols, respectively.

FIG. 3. Similarity matrix for deduced Arabidopsis and poplar SOD amino acid sequences. Similarities between protein sequences were calculated based on pairwise alignments using the EMBOSS Pairwise Alignment Algorithms (www.ebi.ac.uk/Tools/emboss/align/).

FIG. 4. Proposed exon sequences for PtFSD2.2 after manual curation using the PtFSD2.1 gene model found in Phytozome. Nucleotide insertions are shown in shade. The premature stop codon is underlined in exon six. Sequences are SEQ ID NOs: 47-55, from top to bottom.

FIG. 5. Gene structure (exons and introns) of Populus trichocarpa and Arabidopsis thaliana SODs and CCSs. A. Gene structure for CSDs and CCSs. B. Gene structure for MSDs and FSDs. Exons, shown with squares, and introns shown as lines, are drawn to scale. Similar or equivalent exons based on similarities in their encoding amino acid sequences have the same color within each group (CCSs, CSDs, MSDs and FSDs).

FIG. 6. Relative transcript levels of poplar SODs and CCSs in various tissues under well-watered, drought, and recovery conditions. Transcript levels were measured by RT-qPCR and normalized against three reference genes (see Methods; FIG. S1). Sink leaves (SiL), source leaves (SoL), stems (Stm), main roots (RA) and fine roots (RB) were analyzed. Values represent means of two biological replicates with standard deviations. A two-way ANOVA of observed transcript levels of SOD genes (all tissues vs. water availability) is provided in Table S2.

FIG. 7. Relative transcript abundance of poplar SODs and CCSs comparing transgenic GS and wild-type poplars under well-watered, drought or recovery conditions. Values represent the log ratio of transcript levels (transgenics/wild type) (RT-qPCR data; as for FIG. 6 for visualization by the Heat Mapper Plus tool (bar.utoronto.ca/welcome.htm). Samples are sorted by conditions (well-watered, drought, and recovery) and by tissue [sink leaves (SiL), source leaves (SoL), stems (Stm), main roots (RA) and fine roots (RB)]. Gene descriptors are colored according to the predicted subcellular localizations (see Table 1) and arranged according to the clustering pattern obtained using the Cluster 3 and Java TreeView programs (see Methods). Genes with significant differences between WT and GS transgenic across tissues under drought stress condition (Table S3) are underlined.

FIG. 8. Whole-genome microarray analysis (Agilent Populus whole genome array; 4×44K platform) of genes differentially expressed between wild type and GS transgenics. Differential expression was determined by p-values adjusted with the SLIM method [74], with a fold-change cut-off of two. Relative expression (log ratio of GS/wild type) in four tissues [sink leaves (SiL), source leaves (SoL), stems (Stm) and main roots (RA)] during drought was shown, with red indicating up-regulation and blue, down-regulation in the GS transgenics. Two biological replicates were included. Genes annotated as superoxide dismutase are listed in bold.

FIG. 9. SOD activities as detected by in-gel assays. Total proteins were extracted from source and sink leaves of two transgenic lines (T1 and T2) and wild type control plants (WT) grown under drought conditions. A total of 75 μg protein was loaded per well. Iron and Cu/Zn SOD proteins were identified using specific inhibitors as described by Fridovich [46].

### DETAILED DESCRIPTION OF THE INVENTION

Glutamine synthetase (GS) plays a central role in plant nitrogen assimilation, a process intimately linked to soil water availability. We previously showed that hybrid poplar (Populus tremula×alba, INRA 717-1B4) expressing ectopically a pine cytosolic glutamine synthetase gene (GS 1 a) display enhanced tolerance to drought. Preliminary transcriptome profiling revealed that during drought, members of the superoxide dismutase (SOD) family were reciprocally regulated in GS poplar when compared with the wild-type control, in all tissues examined. SOD was the only gene family found to exhibit such patterns.

In silico analysis of the Populus genome identified 12 SOD genes and two genes encoding copper chaperones for SOD (CCSs). The poplar SODs form three phylogenetic clusters in accordance with their distinct metal co-factor requirements and gene structure. Nearly all poplar SODs and CCSs are present in duplicate derived from whole genome duplication, in sharp contrast to their predominantly single-copy Arabidopsis orthologs. Drought stress triggered plant-wide down-regulation of the plastidic copper SODs (CSDs), with concomitant up-regulation of plastidic iron SODs (FSDs) in GS poplar relative to the wild type; this was confirmed at the activity level. We also found evidence for coordinated down-regulation of other copper proteins, including plastidic CCSs and polyphenol oxidases, in GS poplar under drought conditions.

Both gene duplication and expression divergence have contributed to the expansion and transcriptional diversity of the Populus SOD/CCS families. Coordinated down-regulation of major copper proteins in drought-tolerant GS poplars supports the copper cofactor economy model where copper supply is preferentially allocated for plastocyanins to sustain photosynthesis during drought. Our results also extend previous findings on the compensatory regulation between chloroplastic CSDs and FSDs, and suggest that this copper-mediated mechanism represents a common response to oxidative stress and other genetic manipulations, as in GS poplars, that affect photosynthesis.

### I. DEFINITIONS

Various terms relating to the biological molecules of the present invention are used hereinabove and also throughout the specifications and claims. The terms “substantially the same,” “percent similarity” and “percent identity” are defined in detail below.

With reference to nucleic acids of the invention, the term “isolated nucleic acid” is sometimes used. This term, when applied to genomic DNA, refers to a DNA molecule that is separated from sequences with which it is immediately contiguous (in the 5′ and 3′ directions) in the naturally occurring genome of the organism from which it was derived. For example, the “isolated nucleic acid” ray comprise a DNA molecule inserted into a vector, such as a plasmid or virus vector, or integrated into the genomic DNA of a prokaryote or eukaryote. An “isolated nucleic acid molecule” may also comprise a cDNA molecule or a synthetic DNA molecule.

With respect to RNA molecules of the invention, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form.

Nucleic acid sequences and amino acid sequences can be compared using computer programs that align the similar sequences of the nucleic or amino acids thus define the differences. For purposes of this invention, the GCG Wisconsin Package version 9.1, available from the Genetics Computer Croup in Madison, Wis., and the default parameters used (gap creation penalty=12, gap extension penalty=4) by that program are the parameters intended to be used herein to compare sequence identity and similarity.

The term “substantially the same” refers to nucleic acid or amino acid sequences having sequence variations that do not materially affect the nature of the protein (i.e. the structure, thermostability characteristics and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

The term “ectopic expression” refers to a pattern of subcellular, cell-type, tissue-type and/or developmental or temporal (e.g., light/dark) expression that is not normal for the particular gene or enzyme in question. Such ectopic expression does not necessarily exclude expression in normal tissues or developmental stages.

The term “overexpression” means a greater than normal expression level of a gene in the particular tissue, cell and/or developmental or temporal stage for the gene. Such overexpression results in “overproduction” of the enzyme encoded by the gene, which means a greater than normal production of the enzyme in a particular tissue or cell, or developmental or temporal stage for the enzyme. The terms “underexpression” and “underproduction” have an analogously converse meaning, and are used interchangeably with the term “suppression”.

In regards to the present invention, “equivalent plants” are ones of the same genotype or cultivar, at the same age, and having been grown under the same conditions. In the case where one is a transgenic plant, the equivalent plant may be transformed by a similar DNA construct but without the glutamine synthetase transgene, or may not be transformed but regenerated from tissue culture.

In this invention, the term “promoter” or “promoter region” refers to the 5′ regulatory regions of a gene, including promoters per se (e.g., CaMV 35S promoters and/or tetracycline repressor/operator gene promoters), as well as other transcriptional and translational regulatory sequences.

The term “selectable marker” refers to a gene product that confers a selectable phenotype, such as antibiotic resistance, on a transformed cell or plant. Selectable markers are encoded by expressible DNA sequences, which are sometimes referred to herein as “selectable marker genes.”

The terms “operably linked”, “operably inserted” or “operably associated” mean that the regulatory sequences necessary for expression of the coding sequences are placed in the DNA molecule in the appropriate positions relative to the coding sequence so as to effect expression of the coding sequence. This same definition is sometimes applied to the arrangement of coding sequences and transcription control elements (e.g. promoters, enhancers, and termination elements) in an expression vector.

The phrase “DNA construct” refers to genetic sequence used to transform plant cells and generate progeny transgenic plants. At minimum a DNA construct comprises a coding region for a selected gene product, operably linked to 5′ and 3′ regulatory sequences for expression in transformed plants. In preferred embodiments, such constructs are chimeric, i.e., the coding sequence is from a different source one or more of the regulatory sequences (e.g., coding sequence from tobacco and promoter from cauliflower mosaic virus). However, non-chimeric DNA constructs also can be used.

DNA constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in Ausubel et al. (1998). A plant species or cultivar may be transformed with a DNA construct (chimeric or non-chimeric) that encodes a polypeptide from a different plant species or cultivar (e.g., poplar transformed with a gene encoding a pine protein). Alternatively, a plant species or cultivar may be transformed with a DNA construct (chimeric or non-chimeric) that encodes a polypeptide from the same plant species or cultivar. The term “transgene” is sometimes used to refer to the DNA construct within the transformed cell or plant. Transgenic poplar plants have been generated using a LR Gateway reaction which results in insertion of the transgene into the destiny vector pGWB2 (a gift from Tsuyoshi Nakagawa). Then, a DH5a strain was transformed to select plasmids with the transgene (YEP+Hyg50+Kan50). Selected plasmids were used to transform Agrobacterium strain (C53C8 pTOK47, Rifampicin and carbenicillin resistant) and the selection was then made in YEP+Hyg50+Kan50+Cb 100+Rif 50.

In accordance with the present invention, nucleic acids having the appropriate sequence homology with the nucleic acids of the invention may be identified by using hybridization and washing conditions of appropriate stringency. For example, hybridizations may be performed, according to the method of Sambrook et al. (1989, Molecular Cloning, Cold Spring Harbor Laboratory), using a hybridization solution comprising: 5×SSC, 5×Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology (Sambrook et al., 1989, supra):

As an illustration of the above formula, using [Na+]=[0.368] and 50% formamide, with GC content of 42% and an average probe size of 200 bases, the Tm is 57° C. The Tm of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tm of the hybrid. Wash conditions should be as stringent as possible for the degree of identity of the probe for the target. In general, wash conditions are selected to be approximately 12-20° C. below the Tm of the hybrid in regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 mg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

### II. DESCRIPTION

We describe the characterization of three poplar genes that are associated with drought tolerance in transgenic poplar plants expressing ectopically the pine cytosolic glutamine synthetase (GS) gene. Glutamine synthetase (GS) plays the central role in assimilation of ammonium into amino acids and other reduced nitrogen compounds in plants. Hybrid poplar (Populus tremula×P. alba, INRA 717-1-B4) expressing ectopically the pine glutamine synthetase (GS 1 a) gene display pleiotropic phenotypes, including increased growth, increased nitrogen use efficiency, and enhanced tolerance to drought. This prompted us to profile transcriptomic changes associated with GS overexpression during pre-drought, drought, and recovery conditions in poplar tissues using microarrays (Agilent Populus whole genome array; 4×44K platform) and qPCR validations of candidate genes. Under drought conditions a shift was seen in the percentage of differentially expressed genes in transgenics (drought tolerant) with regard to wild type controls (sensitive to drought). Among up-regulated genes, the stress group was one of the most significant. Three specific genes, PtFSD2.1, PtFSD3 and PtYSL showed at least 2-fold higher expression (SLIM 2×) in GS poplars than in wild type control plants in all four tissues investigated (sink leaves, source leaves, stems, and roots) under drought conditions. Enhanced expression of these genes in all tissues of drought-tolerant poplar is clearly correlated with drought tolerance. Thus, these genes can serve as significant markers for drought tolerance in marker-assisted selection of drought resistant/tolerant genotypes.

Accordingly, the present invention provides the means to select for drought resistant plants. In another aspect, a transgenic woody perennial plant exhibiting altered expression levels of at least one gene selected from PtFSD2.1, PtFSD3 and PtYSL is provided. These plants exhibit altered stress responses, particularly to drought and oxidative stress. In particular, the invention relates to altering the activity of enzymes involved in drought stress resistance in order to engineer trees with better growth characteristics, higher biomass production, less requirement for fertilizer, better nutritional qualities, and/or improved seed or fruit yield.

A particularly preferred embodiment of the invention comprises poplar trees engineered to ectopically over-express or under express members of the superoxide dismutase gene family.

Provided in accordance with the present invention is an expression cassette for altering the level of at least one superoxide dismutase in plant cells which optionally overexpresses glutamine synthetase 1 (GS1a). In another embodiment the cell expresses both GS1a and at least one SOD. The expression cassette can be used to manipulate stress responses in plants. In a preferred embodiment, the expression cassette comprises the coding sequence of a gymnosperm superoxide dismutase gene operably linked to a promoter.

In another preferred embodiments, the expression cassette contains sequences that are similar to the to the pine SOD coding sequence. Because each amino acid is encoded by several codons, a protein identical to Pinus sylvestris SOD may be encoded by many different coding sequences. Additionally, proteins have a similar enzymatic function to SOD and yet have a different amino acid sequence through the substitution of structurally similar amino acids. Therefore coding sequences that are similar yet not identical to Pinus sylvestris SOD are contemplated in regards to the present invention. In a preferred embodiment, the expression vector comprises a nucleic acid sequence is at least 85% identical to the SOD sequences disclosed herein.

Expression cassettes for expressing a DNA sequences in selected plant cells comprise a DNA sequence of interest operably linked to appropriate 5′ (e.g., promoters and translational regulatory sequences) and 3′ regulatory sequences (e.g., terminators). In a preferred embodiment, the coding region of a gymnosperm SOD gene is placed under a powerful constitutive promoter, such 8 the Cauliflower Mosaic Virus (CaMV) 35S promoter. Other constitutive promoters contemplated for use in the present invention include, but are not limited to: figwort mosaic virus 35S promoter, T-DNA mannopine synthetase, nopaline synthase (NOS) and octopine synthase (OCS) promoters.

Expression cassettes that express a gymnosperm SOD coding sequence under an inducible promoter (either its own promoter or a heterologous promoter) are also contemplated to be within the scope of the present invention. Inducible plant promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, gluconase genes, chitinase genes, etc.) and dark-inducible gene promoters (e.g., asparagine synthetase gene) to name a few.

Organelle-specific, tissue-specific, and development-specific promoters are also contemplated for use in the present invention. Examples of these included, but are not limited to: the ribulose bisphosphate carboxylase (RuBisCo) small subunit gene promoters or chlorophyll a/b binding protein (CAB) gene promoters for expression in photosynthetic tissue; the various seed storage protein gene promoters for expression in seeds; and the root-specific glutamine synthetase gene promoters where expression in roots is desired. Examples of organelle specific promoters include, but are not limited to the ribulose bisphosphate carboxylase (RuBisCo) large subunit gene promoter and the D1 protein promoter. In a preferred embodiment, the expression cassette comprises a chloroplast specific promoter.

Expression cassettes that down-regulate or inhibit expression of SOD are also contemplated in accordance with the present invention. This may be necessary in order to divert nitrogen assimilation or utilization to an alternative pathway, e.g., an engineered pathway that is more efficient than the natural pathway. To accomplish this, the gymnosperm SOD coding sequence or a fragment thereof may be utilized to control the production of the encoded protein. In one embodiment, full-length antisense molecules or antisense oligonucleotides, targeted to specific regions of the encoded RNA that are critical for translation, are used. The use of antisense molecules to decrease expression levels of a pre-determined gene is known in the art. In a preferred embodiment, the expression cassette expresses all or part of the antisense strand of a SOD coding sequence. In another embodiment, an expression cassette that causes the overexpression of the gene targeted for down-regulation is induced to generate a co-suppression effect. In another embodiment, an expression cassette for down-regulation of the SOD enzyme comprises a sequence that encodes a SOD with mutations in the active site of enzyme.

In some instances, it may be advantageous to engineer the expression cassette such that it encodes a “transit” sequence enabling the encoded SOD to cross the chloroplast membrane and localize within the chloroplast. Certain genes naturally comprise such a transit sequences. Cytosolic isozymes, such as SOD, can be targeted to the chloroplast through the in-frame inclusion of a DNA segment encoding such a transit sequence, according to known methods. This expression cassette may be of particular utility in production of transgenic gymnosperms.

The coding region of the expression cassette is also operably linked to an appropriate 3′ regulatory sequence. In a preferred embodiment, the nopaline synthetase polyadenylation region (NOS) is used. Other useful 3′ regulatory regions include, but are not limited to the octopine (OCS) polyadenylation region.

Also provided in accordance with the present invention is a vector containing the expression cassette of the invention. This vector may be used to maintain the expression cassette in bacteria, such as Echerichia coli. Vectors that may be used to maintain the expression cassette in E. coli are well known to those in the art. The expression cassette may also have a more specialized function of introducing the expression cassette into a plant cell. These vectors may be specialized for the various well known ways of introducing transgenes into plant cells. Vectors that may be used for chloroplast transformation are contemplated in regards to the present invention. Examples of vectors for chloroplast transformation include, but are not limited to, pZS197 (Svab and Maliga, 1993, PNAS 90:915-917). In a most preferred embodiment, the vector contains the nucleic acid sequences needed to allow the expression cassette to be stably inserted into the genome of the desired woody perennial by Agrobacterium tumefaciens-mediated plant transformation.

In a preferred embodiment, the vector is an Agrobacterium binary vector. Such vectors include, but are not limited to, BIN19 (Bevan, 1984, Nucleic Acid Res 12: 8711-8721) and derivatives thereof, the pBI vector series (Jefferson et al., 1987, PNAS 83:8447-51), and binary vectors pGA482 and pGA492 (An, 1986) and others (for review, see An, 1995, Methods Mol Biol 44:47-58). In a particularly preferred embodiment, the vector is pBIN19 (Bevan, 1984, Nucleic Acid Res 12: 8711-8721).

Using an Agrobacterium binary vector system, the aforementioned expression cassette is linked to a nuclear drug resistance marker, such as kanamycin. In a preferred embodiment, the neomycin phosphotransferase II gene from pCaMVNEO is used (Fromm et al., 1986, Nature 319: 791-793). Other useful selectable marker systems include, but are not limited to: other genes that confer antibiotic resistances (e.g., resistance to hygromycin or bialaphos) or herbicide resistance (e.g., resistance to sulfonylurea, phosphinothricin or glyphosate).

Also provided in accordance with the present invention is a method to make a woody perennial plant with altered concentrations of SOD in its cells. This method comprises the step of stabily integrating the expression cassette with the SOD coding sequence into the genome of a woody perennial plant cell. Several ways to integrate a transgene such as the expression cassette into a plant cell genome are possible, including but limited to, Agrobacterium vectors, PEG treatment of protoplasts, biolistic DNA delivery, UV laser microbeam, gemini virus vectors, calcium phosphate treatment of protoplasts, electroporation of isolated protoplasts, agitation of cell suspensions with microbeads coated with the transforming DNA, direct DNA uptake, liposome-mediated DNA uptake and chloroplast transformation (Maliga et al., 1995, U.S. Pat. No. 5,451,513). Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratory Manual (Maliga, Klessig, Cashmore, Gruissem & Varner, eds., 1994). In a preferred embodiment, Agrobacterium-mediated transformation is used.

Agrobacterium-mediated transformation of plant nuclei is accomplished according to the following procedure:

(1) the gene is inserted into the selected Agrobacterium binary vector;

(2) transformation is accomplished by co-cultivation of an appropriate plant tissue (such as leaf tissue in poplar) with a suspension of recombinant Agrobacterium, followed by incubation (e.g., two days) on growth medium in the absence of the drug used as the selective medium (see, e.g., Horsch et al., 1985, Cold Spring Harb Symp Quant Biol 50:433-7);

(3) plant tissue is then transferred onto the selective medium to identify transformed tissue; and

(4) identified transformants are regenerated to intact plants.

It should be recognized that the amount of expression, as well as the tissue specificity of expression of the transgenes in transformed plants can vary depending on the position of their insertion into the nuclear genome. Such position effects are well known in the art. For this reason, several transformants should be regenerated and tested for expression of the transgene.

Plants are transformed and thereafter screened for one or more properties, including expression of the transgene, altered responses to stress or drought, higher growth rates, biomass accumulation rates, higher protein or chlorophyll concentration, or changes in growth habit or appearance (e.g., alteration of phyliotaxy and canopy structure—the arrangement of leaves and branches to optimize light reception—alterations of which have been observed in the exemplified transgenic poplar).

Also provided in accordance with the present invention is transgenic woody perennial plant with altered concentrations of SOD in its cells, which exhibits altered stress responses. The successful transformation of poplar (an angiosperm) with a pine (a gymnosperm) GS1 gene and heterologous SOD enzymes, and the greatly improved phenotype obtained thereby, indicates that stress responses may be favorably improved in woody perennials more dramatically than hitherto expected. Accordingly, although in a particularly preferred embodiment the woody perennial is poplar, (specifically hybrid poplar clone INRA 7171-B4, Populus tremula×P. alba), other members of the genus Populus (which includes cottonwood, aspen and poplar) and the family Salicaceae are also preferred for practice of the present invention. In other embodiments, a wide variety of woody perennials are contemplated as targets for similar genetic engineering using the compositions and methods described herein. These include, but are not limited to, angiosperm forest trees, such as eucalyptus, willow (Salix spp.), birch, oak, cherry, maple, yellow or tulip poplar (genus Liriodendron), sweetgum, acacia, teak, Liquidamber spp. and Alnus spp., among others; gymnosperm forest trees, such as pine, spruce, fir, redwood, Douglas fir, Araucaria spp. and Cryptomeria spp., among others; as well as fruit and nut-bearing trees and ornamental trees and shrubs.

Also provided in accordance with the current invention is a poplar tree that has a statistically significant higher growth rate, and higher resistance to drought stress than its untransformed equivalent. In a preferred embodiment, this transgenic tree exhibits at least 10% greater resistance to drought stress during the first 3 months in the greenhouse after transformation as compared to untransformed trees of the same cultivar. More preferably, the transgenic poplar is 40% greater, and in a most preferred embodiment, the transgenic tree is 60% greater.

The preceding description set forth the general procedures involved in practicing the present invention. To the extent that specific materials are mentioned, it is merely for purposes of illustration and is not intended to limit the invention. Unless otherwise specified, general cloning procedures, such as those set forth in Sambrook et al., Molecular Cloning, Cold Spring Harbor Laboratory (1989) or Ausubel et al. (eds.) Current Protocols in Molecular Biology, John Wiley & Sons (1999) are used.

### III. USES FOR THE WOODY PERENNIALS WITH ALTERED DROUGHT STRESS RESPONSES

The genetically modified trees and other woody perennial plants of the present invention are expected to be of use for a variety of agronomic and/or horticultural purposes. For instance, due to their increased resistance to oxidative stress, they may be productively cultivated under nitrogen nutrient deficient conditions (i.e., copper-poor soils and low nitrogen fertilizer inputs) that would be detrimental to the growth of wild-type trees. The engineered trees may also be advantageously used to achieve earlier maturing, faster growing, and/or higher yielding crops and/or produce more nutritious foods (fruit and nuts) and animal fodder when cultivated under nitrogen non-limiting growth conditions (i.e. soils or media containing or receiving sufficient amounts of nitrogen nutrients to sustain healthy tree growth).

The transgenic plants of the invention may be used for plant breeding or directly in silvaculture applications. Plants containing one transgene may be crossed with plants containing a complementary transgene in order to produce plants with enhanced or combined phenotypes.

The following materials and methods are provided to facilitate the practice of the present invention.

### Plant Materials and Stress Treatments

Hybrid poplar (Populus tremula×P. alba, INRA 717-1B4) expressing ectopically the pine glutamine synthetase gene (GS 1 a) were generated and maintained as previously described [7]. Water stress treatments and conditions of recovery from water stress were as described in El-Khatib et al. [11]. Rooted cuttings (9-12 months old) were planted in 6-inch pots containing a peat-based commercial growth medium (Metro-Mix 200, Scotts, Marysville, Ohio) without supplementary nutrients and raised in a growth chamber supplying a 16 h photoperiod (24-26° C.). Soil samples were weighed after drying overnight at 60° C. and volumetric soil moisture contents (θ) were calculated. Nonlinear regression (SigmaPlot v4.01, SPSS, Chicago, Ill.) was used to relate θ to soil water potential (ψsoil): ψsoil=0.9031+1.305 ln(θ−0.1081) (R2=0.98; P, 0.0001). This allowed conversion of θ, estimated with a time-domain-reflectrometry (TDR) soil moisture meter (Theta Meter, Delta-T Devices, Cambridge, U.K.), to track changes in soil water throughout the experiment. We used soil water potential as a proxy measure of plant water status. Plants were watered every day until θ was between 50 and 55%, equivalent to a soilwater potential of −1 to 0 MPa for well-watered conditions. Drought stress was applied to plants by withholding irrigation for 7 days, by which time θ was between 15 and 20%, equivalent to a soil water potential of −2 to −3 MPa. This level of water stress typically resulted in a decline in leaf stomatal conductance in wild type poplars from 0.138 mol m⁻²s⁻¹ (SE 0.025) for well-watered leaves to 0.018 mol m⁻²s⁻¹ (SE 0.002) during drought conditions (unpublished data). After the drought treatment, plants were watered every day for 5 days recovering the well-watered conditions in soil. Plants heights ranged from 45 to 55 cm at the collection day.

### Sequence Analysis

Published Arabidopsis and Populus SODs (NCBI) were used to search the P. trichocarpa genome v2.2 (www.phytozome.net) by BLAST [28]. Open reading frames, exon-intron predictions, and 3′-UTRs were manually examined and analyzed against publicly available poplar ESTs. Theoretical molecular weights and isoelectric points for the predicted proteins were calculated using the Expasy server (expasy.org/tools/pi_tool.html) [29]. Pairwise sequence similarities were calculated individually using the EBI EMBOSS Pairwise Sequence Alignment server (www.ebi.ac.uk/Tools/emboss/align/). The similarity of a group was calculated as the mean of all individual pairwise comparisons within that group. The similarity between groups was calculated as the mean of all between-group pairwise comparisons.

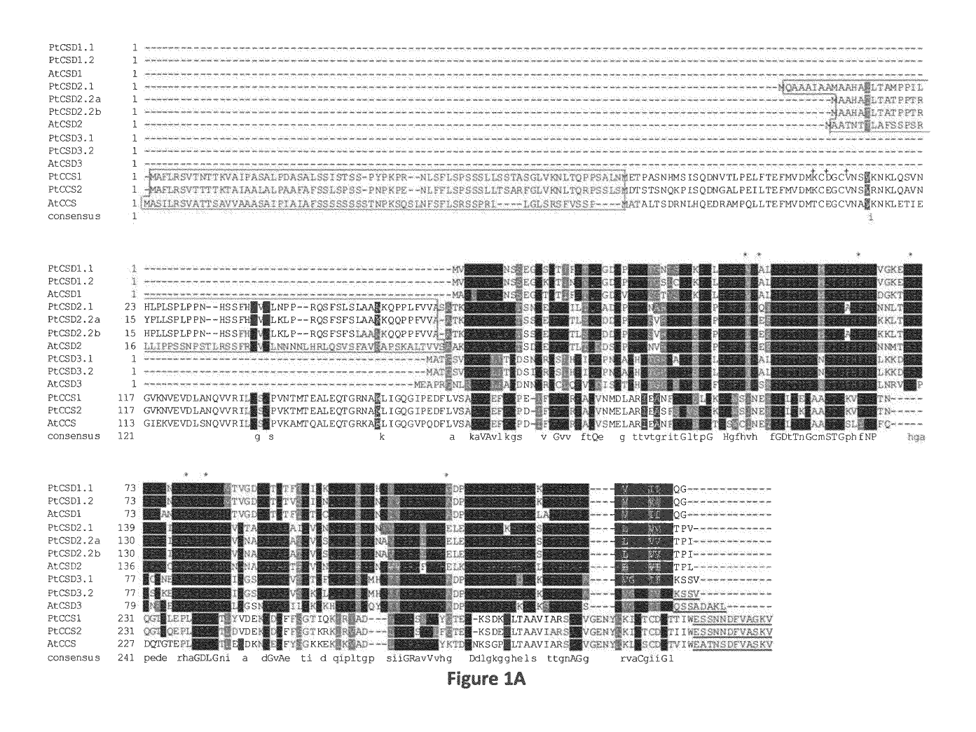

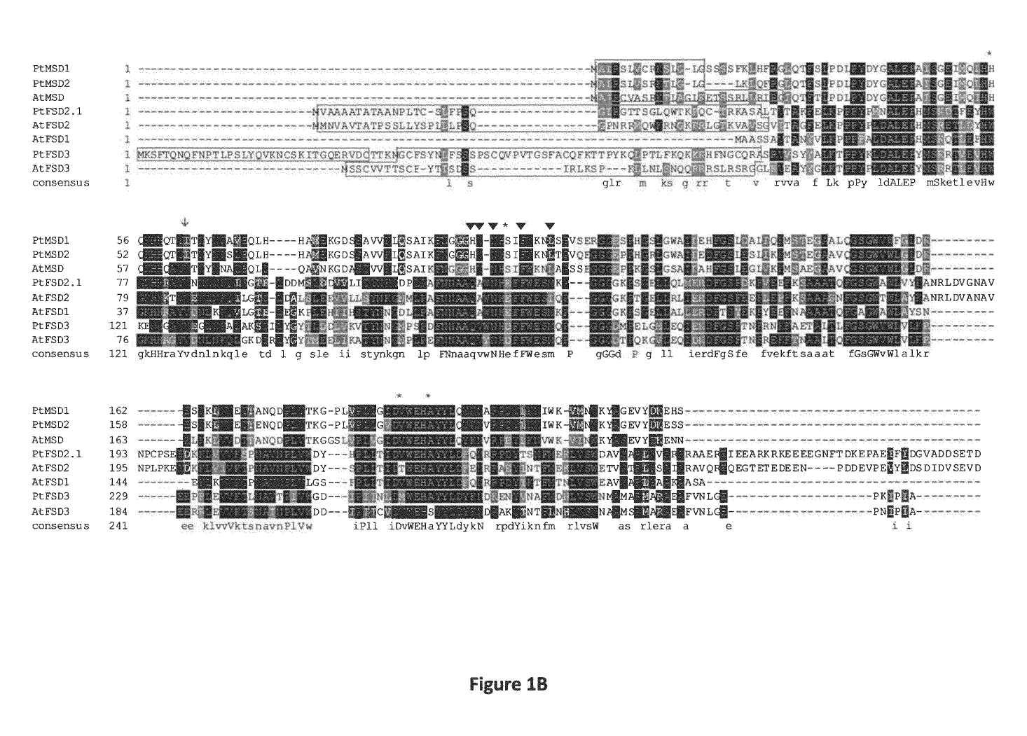

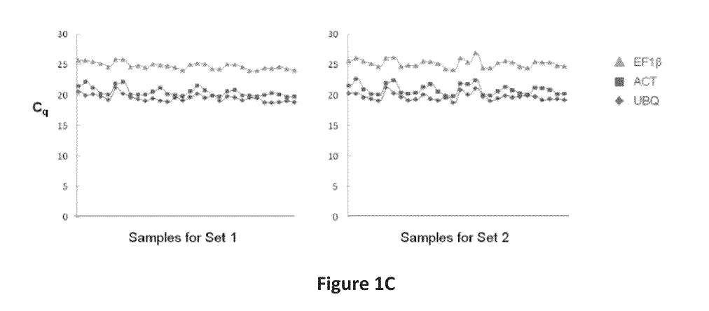

The alignments in FIG. 1 were prepared using ClustalX 2.0.12 [30]. Boxshade 3.21 (www.ch.embnet.org/software/BOX_form.html) was used to mark identity and similarity boxes and consensus lines in amino acid alignments. The Neighbor joining tree was constructed using the Muscle alignment program implemented in MEGA version 5 [31], with partial deletion to handle alignment gaps, and 1000 bootstrap iterations. Poplar SOD gene nomenclature in this paper was assigned considering its phylogenetic relationship with the published nomenclature for the Arabidopsis SOD gene family [15].

TargetP 1.1 [32] (www.cbs.dtu.dk/services/TargetP/) was used for general subcellular localization prediction of poplar SODs and CCSs. Following the recommendation of Emanuelsson et al. [32], proteins predicted as “other” (other than chloroplast, mitochondria or secreted) by the TargetP 1.1 were further analyzed by TMHMM 2.0 (www.cbs.dtu.dk/services/TMHMM/) to assess transmembrane helices. Sequences predicted as “secretory” or had low reliability (RC>4) were further analyzed using SignalP 4.0 [33] (www.cbs.dtu.dk/services/SignalP/). ChloroP 1.1 [34] (www.cbs.dtu.dk/services/ChloroP/) and MITOPROT [35] were used to produce a detailed report for chloroplast- and mitochondria-targeted proteins, respectively. PTS 1 [36] (www.mendel.imp.ac.at/mendeljsp/sat/pts1/PTS1predictor.jsp)) was used for peroxisomal protein predictions.

qPCR

RNA extraction was carried out as described in Liao et al. [37]. RNA was extracted from two biological replicates consisting of pooled samples from 5 individual plants from 2 replicate experiments. Each experiment assessed the GS transgenic line (line 4-29) and the wild type control. Quality of the RNA was assessed both on agarose gels and spectrophotometrically. Although no contamination by genomic DNA was detected on gels, all RNA samples were treated with DNases (Turbo DNA Free kit of Applied Biosystems/Ambion, Austin Tex.), following the manufacturer's protocol, and stored at −80° C. for up to three months. For cDNA synthesis, the iScript Select cDNA Synthesis kit (Bio-Rad, Hercules, Calif.) was used with both random and oligo dT primers using 3 μg of total RNA per reaction (80 μL), according to the manufacturer's instructions. cDNAs were stored at −20° C. for up to six months.

Quantitative PCR was performed using a LightCycler 480 (Roche Applied Science, Indianapolis Ind.) using Roche SYBR Green I Master mix prepared according to the manufacturer's specifications. qPCR reactions were carried out in 20 μL volumes containing 10 ng cDNA and 0.5 μM primers. A total of 45 cycles were run per program: denaturing was at 95° C. for 10 sec, annealing at 58° C. for 15 sec, and extension was at 72° C. for 12 seconds in each cycle.

P. trichocarpa genome sequences and Populus EST sequences (P. tremula and P. alba) were used in the design of the primers for qPCR (Table S1). The forward primers were designed within the coding regions and the reverse primers were designed in 3′UTRs. Primer quality was evaluated using Prime3Plus (www.bioinformatics.nl/cgi-bin/primer3plus/) [38]. All amplicons were between 155 and 305 bp. Sequences of the resulting amplicons were validated by sequencing the RT-qPCR product. Relative transcript levels were determined against three validated reference genes: actin, elongation factor 1b and ubiquitin [39], using GeNorm [40] (FIG. S1). Quantitative cycles were estimated using LinRegPCR (v 11.1) [41]. In all cases, two biological replicates were used, each with three technical replicates. Cluster 3.0 [42] and Java TreeView [43] programs were used as the computational and graphical environment for analyzing correlations from RT-qPCR expression data. The heat map was generated using Heat Mapper Plus (Bio-Array Resource for Plant Biology; bar.utoronto.ca/welcome.htm).

### Determination of SOD Activities

In order to provide assessment of qualitative differences in activities of the various SODs in GS transgenic and control leaves, proteins were extracted from three biological replicates (individual plants) in two replicate experiments and on native protein gels. Proteins were extracted by mixing one part of liquid nitrogen-ground tissue with two parts of extraction buffer [50 mM KH₂PO4 pH 7.8, 1 mM EDTA, 0.1% (w/v) Triton X-100, and 0.05% (v/v) b-mercaptoethanol] and incubated on ice for 10 min. Samples were centrifuged at 13,000 g for 12 min at 4° C. and protein concentrations were determined spectrophotometrically [44] using BSA as a standard. The protocol of Weydert and Cullen [45] was followed to assess SOD activities using native gels (acrylamide and bis-acrylamide solution (29:1) 12%, w/v; 1.5 mm thickness) with slight modifications. Gels were first run at 20 mA for one hour, followed by 30 mA for two hours, after which the electrophoresis buffer was replaced. The gels were then run at 40 mA for 20 min after run-off of the dye front. Seventy-five micrograms total protein was found optimal for protein separation. Assays of the three SOD activities (Cu/ZnSODs, MnSODs, and FeSODs) were performed using specific inhibitors (KCN and H₂O₂), as previously described [46]. Gels were scanned, negative images were obtained, and intensities of bands were measured using Image J 1.43 [47].

The following example is provided to illustrate certain embodiments of the invention. It is not intended to limit the invention in any way.

### Example I

### The Populus Superoxide Dismutase Gene Family and its Responses to Drought Stress in Transgenic Poplar Overexpressing a Pine Cytosolic Glutamine Synthetase (GS1a)

Considering the relevant role of the SODs in drought tolerance, we have undertaken in silico characterization of the SOD gene family in poplar and assessed transcript levels for the SOD gene family in various tissues of GS transgenic and wild type poplars subjected to drought treatments. Furthermore, we have detected the activities of the major poplar SODs in gel assays. Our results show that drought tolerant GS poplars have altered SOD expression when compared with the wild type under drought conditions. The putative roles of the poplar SOD gene family and the use of specific SODs as marker(s) of drought tolerance are proposed.

### In Silico Characterization of the SOD Gene Family in Populus

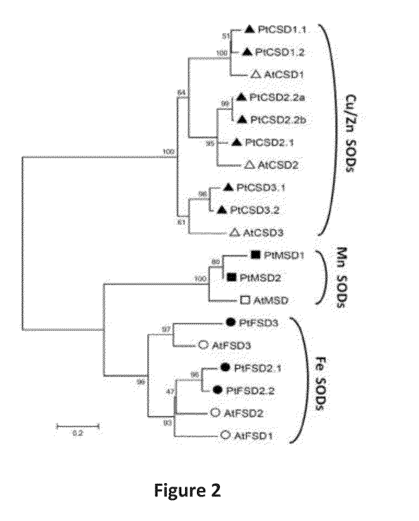

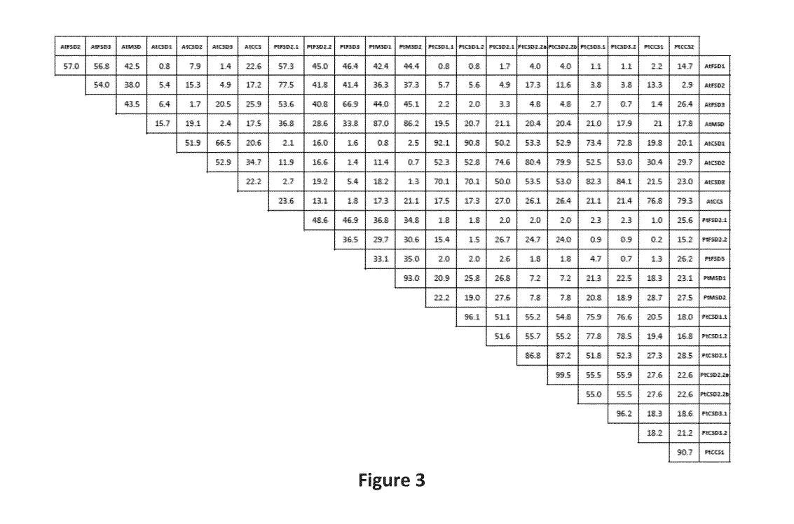

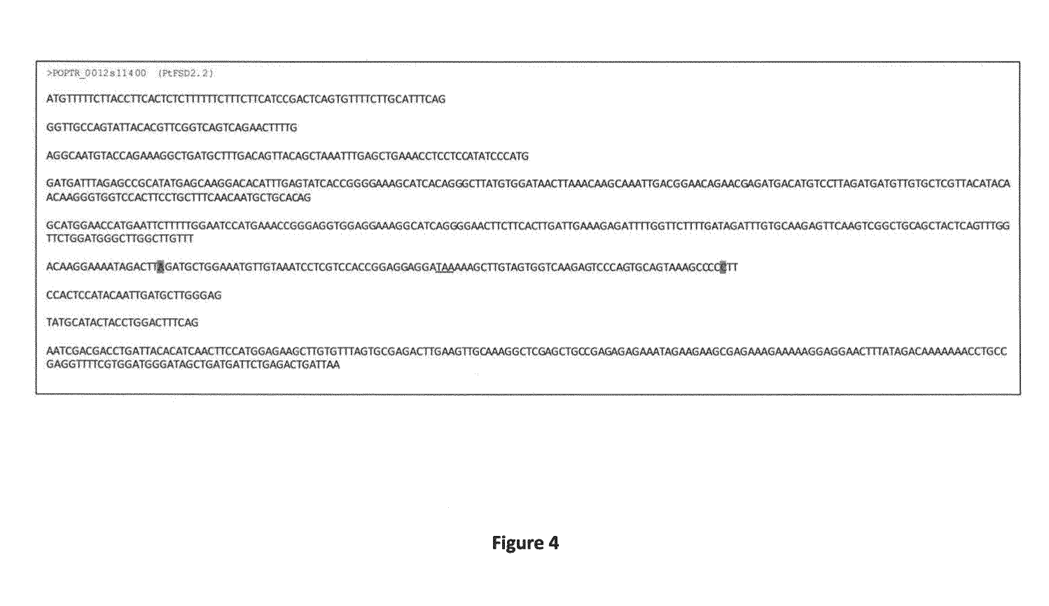

Twelve putative SODs were identified in the P. trichocarpa genome (Phytozome) by BLAST using Arabidopsis and poplar sequences functionally annotated as SODs in the NCBI database as queries. To propose a nomenclature for the poplar SOD gene family, a phylogenetic tree was constructed using predicted amino acid sequences from Populus and Arabidopsis (FIG. 2). Arabidopsis is the only plant for which the SOD gene family has been fully characterized [15]. In Arabidopsis the SOD family consists of seven members: three Cu/ZnSODs (AtCSDs), one MnSOD (AtMSD), and three FeSODs (AtFSDs). The three groups formed separate clusters in the phylogenetic tree with strong bootstrap support, in accordance with their distinct metal cofactor requirements (FIG. 2). Seven poplar SODs were classified as Cu/ZnSODs in three strongly supported sub-groups (PtCSD1, PtCSD2, and PtCSD3) corresponding to their putative Arabidopsis orthologs. The PtCSD1 sub-group contains two highly similar isoforms, PtCSD1.1 and PtCSD1.2 (96.1% amino acid sequence similarity, FIG. 3), derived from the recent (Salicoid) whole-genome duplication [48] (Plant Genome Duplication Database; chibba.agtec.uga.edu/duplication/). They share high similarity (91-92%) to the putative ortholog, AtCSD1 (FIG. 3). The PtCSD2 sub-group contains three SODs, PtCSD2.1, PtCSD2.2a and PtCSD2.2b, two of which are nearly identical (PtCSD2.2a and PtCSD2.2b; 99.5% similarity). PtCSD2.1 and PtCSD2.2b (87.2% similarity) were derived from the Salicoid whole-genome duplication (Plant Genome Duplication Database), whereas PtCSD2.2a likely originated from PtCSD2.2b via an independent duplication event. The PtCSD2s share 75-80% amino acid sequence similarity with the Arabidopsis ortholog, AtCSD2. The third sub-group also contains a genome duplicate, PtCSD3.1 and PtCSD3.2, with high similarity with one another (96.2%) and with the Arabidopsis AtCSD3 (82-84%). The MnSOD group is the smallest of the three, with two poplar members, PtMSD1 and PtMSD2 (93.0% similarity), derived from genome-wide duplication. They share 86-87% similarity with their Arabidopsis ortholog AtMSD. The FeSOD group contains equal numbers of Populus and Arabidopsis SODs in two sub-clusters. One poplar isoform grouped with AtFSD3 (66.9% similarity) with very strong bootstrap support, and was designated PtFSD3. The other two were derived from genome-wide duplication; one appeared to be a partial sequence. The full-length isoform (POPTR_{—}0015s12190) was most similar to AtFSD2 (77.5%: FIG. 3), thus designated PtFSD2.1, whereas the truncated gene model (POPTR_{—}0012s11400) was named PtFSD2.2. Manual inspection identified five miss-annotated introns and five exons (FIG. 4). The curated gene model contained nine exons (versus four in the Phytozome-predicted model), similar to PtFSD2.1. However, one of the exons in PtFSD2.2 harbored two single-nucleotide insertions relative to PtFSD2.1 (shaded residues in FIG. 4), the first of which led to a premature stop codon. This suggests that PtFSD2.2 may represent a pseudogene. The lone member AtFSD1 shares 57% amino acid sequence similarity with AtFSD2, and they were derived from an older, Brassicaceae-specific (β) duplication event (Plant Genome Duplication Database). Consistent with this, no apparent Populus ortholog of AtFSD1 was identified.

Copper chaperones for Cu/ZnSODs (CCS) were included in this work, since CCS are required for Cu/ZnSOD activity in Arabidopsis [49]. Two putative CCSs homologous to the Arabidopsis AtCCS were identified in the Populus genome, and were designated PtCCS1 and PtCCS2. They appear derived from whole-genome duplication, and shared 90.7% similarity with each other, and 77-79% with AtCCS (FIG. 3). Like several of the SODs, transcript levels of both CCS genes were significantly altered in the GS poplar relative to the wild type under drought, based on our microarray studies (data not shown).

Taken together, our analysis showed that multiple gene duplication events contributed to the expansion of the Populus SOD and CCS families. This resulted in the overall greater numbers of poplar genes in each SOD/CCS group than the number of orthologs found in Arabidopsis, except for the iron SOD group.

### Gene Structure of Populus and Arabidopsis SODs and CCSs

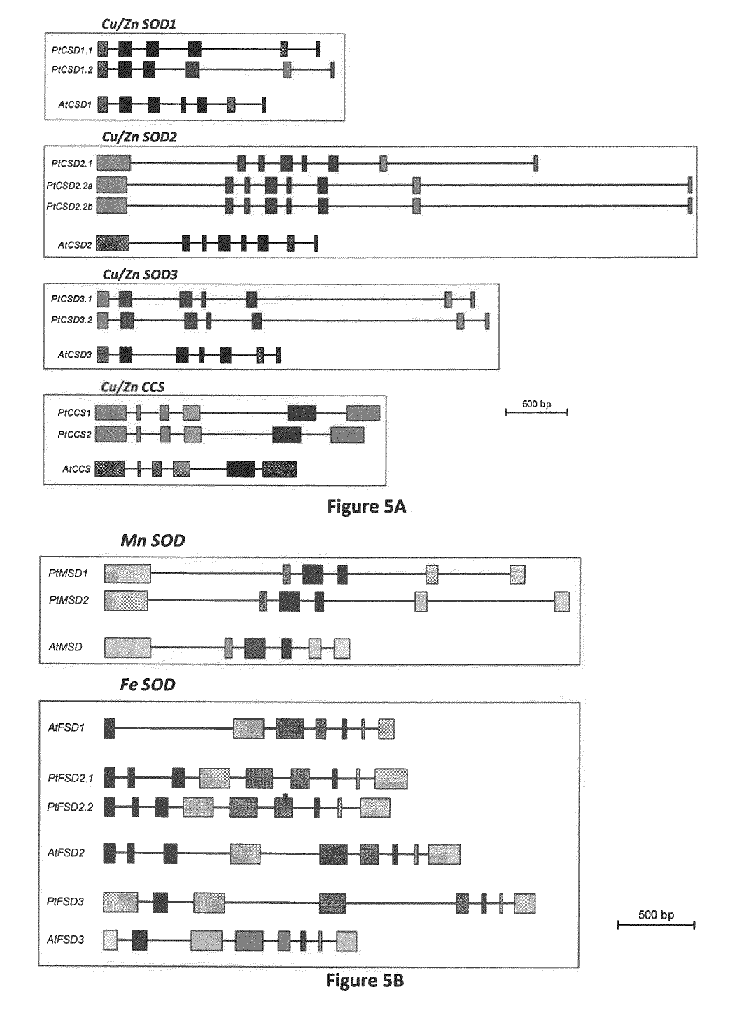

The exon-intron structure was largely conserved among Populus and Arabidopsis Cu/Zn SOD genes, with two exceptions. The exons 4 and 5 were fused in PtCSD1.1 and PtCSD1.2, whereas the second exon was split into two in the CSD2 group (FIG. 5A). The length of exon 1 in the CSD2 group is more than twice as long as exon 1 in the other Cu/ZnSOD groups, due to the presence of putative chloroplast targeting sequence (see below). The gene structure of CCSs is distinct from that of the Cu/ZnSODs, but is conserved between Populus and Arabidopsis (FIG. 5A). The poplar and Arabidopsis MnSOD genes have similar structures (FIG. 5B). Gene structure conservation between Populus and Arabidopsis was also observed for the FeSOD genes, except for the 5′ region that differed among the subgroups (FIG. 5B). The lone AtFSD1 is the shortest, lacking any putative subcellular targeting sequence (see below), perhaps consistent with its origin from a lineage-specific duplication event. Relative to FSD1, FSD2 genes contain two additional exons, and FSD3 genes, one, at the 5′-end. Across all SOD/CCS groups, many of the introns were longer in the Populus genes than in the Arabidopsis homologs, consistent with the genome-wide trend reported earlier [50].

### Conserved Sequence Motifs and Subcellular Localization Prediction

In order to assess conservation of key amino acids for active sites and metal binding domains in the poplar SODs and CCSs, the sequences were divided into two groups for alignment: the Cu/Zn binding group including Cu/ZnSODs and CCSs (FIG. 1A), and the manganese and iron binding group (FIG. 1B). In both groups, all residues previously shown to be involved in metal cofactor binding [51]-[53] are conserved in the poplar proteins (the truncated PtFSD2.2 was excluded from this analysis).

The N-terminal regions were less conserved in both groups, harboring putative transit peptides for subcellular targeting. Several programs, including TargetP 1.1 (for multi-compartments prediction [32]), ChloroP 1.1 (for chloroplastic targeting, [54]), MITOPROT (for mitochondrial prediction [55]), and the PTS1 predictor (for peroxisomal targeting signal prediction [46]), were used to predict subcellular localization (Table 1). Within the Cu/ZnSODs, the CSD2 group with extended N-termini (FIG. 1A and FIG. 5A) was predicted to be chloroplast-localized (Table 1). Neither the CSD1 nor CDS3 groups possess recognizable transient peptides for chloroplastic or mitochondrial targeting or secretory proteins. The PTS 1 predictor indicated a possible peroxisomal localization for PtCSD3.2 and AtCSD3, with some level of uncertainty (termed “twilight zone”, see [36]). Thus, PtCSD3.2 and AtCSD3 were predicted to be cytosolic or have predicted peroxisomal targeting, while PtCSD3.1 and the CSD1 group were predicted to be cytosolic (Table 1). Our predictions for the Arabidopsis CSDs are consistent with those reported earlier [15]. The most consistent subcellular prediction for the CCSs was chloroplast, as reported for AtCCS [54]. In addition, the PTS1 predictor classified PtCCS2 and AtCCS as targeted to the peroxisomes, with PtCCS1 receiving a similar prediction in the “twilight zone”. Moreover, the second methionine in the two poplar and the Arabidopsis CCSs is conserved, and it has been suggested as a second translational start site from which a cytosolic isoform can be produced [55]. Thus, the CCS proteins were predicted to be either cytosolic, chloroplastic, or peroxisomal (Table 1).

TABLE 1
Predicted characteristics of SOD and CCS amino acid sequences from
Populus trichocarpa and Arabidopsis thaliana (included are predicted length in amino acids, predicted
isolectric points, (pI) and predicted subcellular location. Asterisks denote marginal confidence on peroxisomal prediction.

| | Length (a.a.) | pI | | | Length (a.a.) | pI | |
|---|---|---|---|---|---|---|---|
| | precursor/mature | precursor/mature | Subcellular | | precursor/mature | precursor/mature | Subcellular |
| Populus | protein | protein | prediction | Arabidopsis | protein | protein | prediction |
| PtCSD1.1 | 152 | 5.6 | Cytosolic | AtCSD1 | 152 | 5.24 | Cytosolic |
| PtCSD1.2 | 152 | 5.47 | Cytosolic | AtCSD2 | 216/155 | 6.49/5.30 | Chlorop. |
| PtCSD2.1 | 219/156 | 6.28/5.49 | Chlorop. | AtCSD3 | 164 | 7.16 | Cytosolic, |
| | | | | | | | Perox*^{.} |
| PtCSD2.2a | 210/155 | 6.39/5.34 | Chlorop. | AtCCS | 320/254 | 5.60/4.94 | Chlorop., |
| | | | | | | | Perox. and |
| | | | | | | | Cytosolic |
| PtCSD2.2b | 210/155 | 6.44/5.34 | Chlorop. | AtMSD | 231/205 | 8.47/6.06 | Mitoch. |
| PtCSD3.1 | 158 | 6.38 | Cytosolic | AtFSD1 | 212 | 6.06 | Cytosolic |
| PtCSD3.2 | 158 | 6.82 | Cytosolic, | AtFSD2 | 305/259 | 4.89/4.52 | Chlorop. |
| | | | Perox.* | | | | |
| PtCCS1 | 323/253 | 5.04/4.71 | Chlorop., | AtFSD3 | 263/222 | 8.62/5.89 | Chlorop. |
| | | | Perox.* | | | | |
| | | | and | | | | |
| | | | Cytosolic | | | | |
| PtCCS2 | 323/253 | 5.47/4.87 | Chlorop., | | | | |
| | | | Perox. | | | | |
| | | | and | | | | |
| | | | Cytosolic | | | | |
| PtMSD1 | 229/215 | 7.24/6.51 | Mitoch. | | | | |
| PtMSD2 | 225/211 | 6.80/6.21 | Mitoch. | | | | |
| PtFSD2.1 | 307/264 | 5.10/4.80 | Chlorop. | | | | |
| PtFSD3 | 308/221 | 8.09/5.25 | Chlorop. | | | | |

All members of the MnSOD group were predicted to be localized in the mitochondria (Table 1). The consensus target prediction for the FeSOD2s and FeSOD3s was chloroplast-targeting (Table 1). The lone AtFSD1 member did not show any transient peptide signal, and was therefore predicted to be cytosolic. Similar predictions for the AtFSDs have been reported [15]. In general, the predicted subcellular localizations, pI values, and amino acid sequence lengths for poplar and Arabidopsis SOD proteins are similar (Table 1).

### Transcript Levels of SOD and CCS Genes in Wild Type and GS Transgenic Poplars

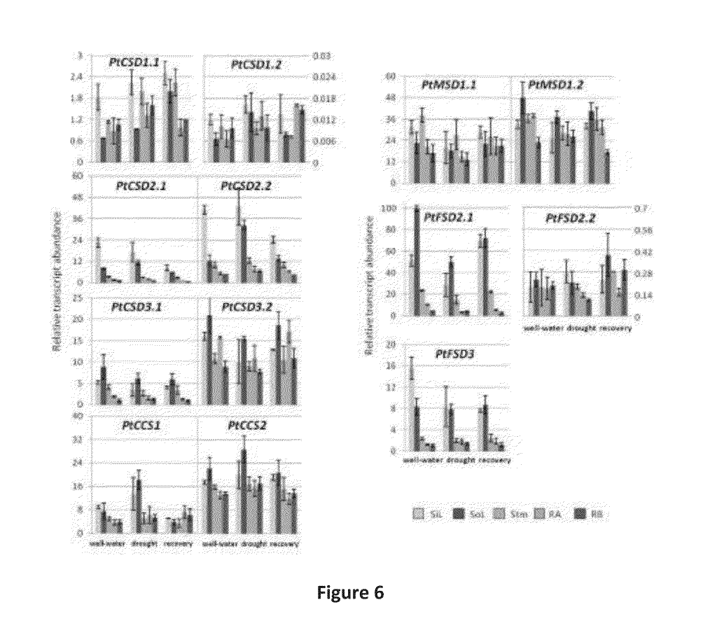

Transcript levels of the poplar SOD and CCS genes were investigated using RT-qPCR. Sink leaves, source leaves, young stem, main roots and fine roots from plants subjected to well-watered, drought and drought recovery conditions were analyzed. Transcripts for all genes were detected in all tissues examined, as shown for the wild type in FIG. 6, although levels of PtFSD2.2 and PtCSD1.2 transcripts were barely detectable (quantification cycles of 30 and 34 in RT-qPCR, respectively), hence they were removed from further analysis. The PtCSD2s, PtFSD2.1 and PtFSD3 exhibited leaf-biased expression across treatments. PtCSD2.2 and PtFSD2.1 were two of the most abundant SOD transcripts in our analysis. PtCSD1.1, PtCCSs and PtMSDs showed no clear tissue specificity. The PtCSD3 pair differed in their tissue distribution patterns, with PtCSD3.1 transcript levels being higher in green tissues than in roots, and PtCDS3.2 showing more uniform transcript levels across all tissues (FIG. 6).

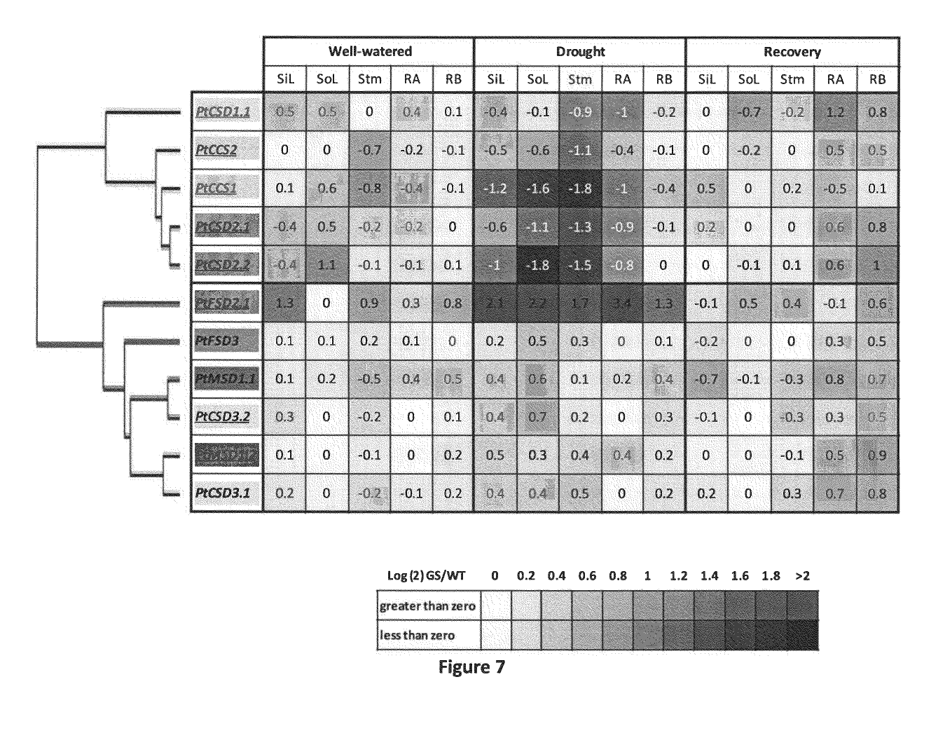

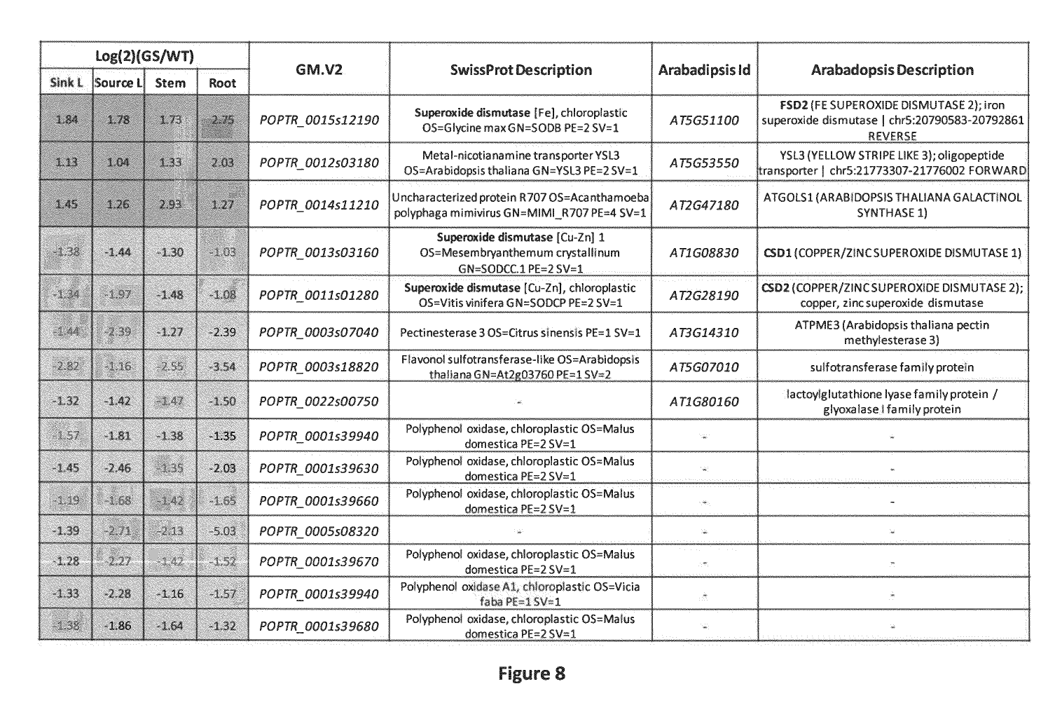

In comparing transcriptional responses to well-watered, drought, and recovery conditions, most SOD/CCS genes showed transcriptional responses to drought compared to the well-watered condition (FIG. 6 and Table S2). Fewer genes showed significant changes in transcript profiles during recovery when compared with the well-watered condition (FIG. 6 and Table S2). In general, greater transcriptional responses were observed in leaves, when compared to other tissues investigated (FIG. 6). Likewise, the response due to GS-overexpression was weak when compared with the wild type under well-watered or recovery conditions (FIG. 7 and Table S3). However, drought stress triggered considerable differences in transcript levels of SOD/CCS genes between wild type and GS poplars (FIG. 7 and Table S3). Cluster analysis revealed two distinct expression patterns (FIG. 7). One group, consisting of PtCSD1.1, PtCSD2s and PtCCSs, showed a clear trend of lower transcript abundance in GS transgenics than in the wild type during drought. The second group consisting of PtCSD3s, PtMSDs and PtFSDs, showed the opposite trend: increased expression in GS transgenics. Consistent with the microarray findings (FIG. 8), the response of PtFSD2.1 (up-regulation in GS poplar) and PtCSD2s (down-regulation in GS poplar) was particularly notable and wide-spread among tissues.

### Altered SOD Activities in Drought-Stressed GS Poplar

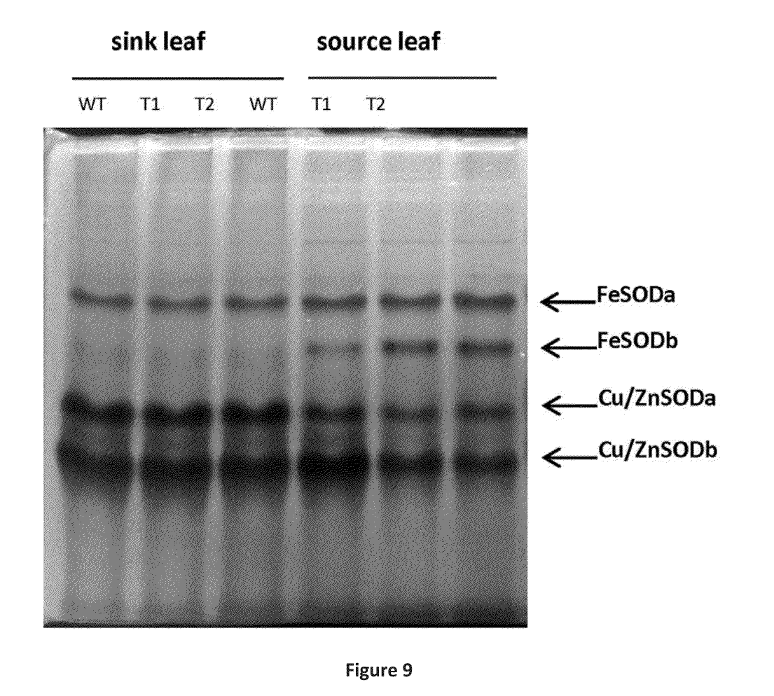

SOD activities were determined by in-gel assays using proteins isolated from leaves of wild type and two GS transgenic lines (FIG. 9). Four main bands showing SOD activity were detected. By using specific inhibitors [46], two bands were confirmed as showing FeSOD activity (FeSODa and FeSODb) and two bands showed Cu/ZnSOD activity (Cu/ZnSODa and Cu/ZnSODb). No consistent differences were observed in SOD activity between transgenic and wild type plants under well-watered conditions (data not shown), but significant differences in SOD activities were detected in drought-stressed source leaves of GS transgenic vs. wild type (FIG. 9). FeSODb, Cu/ZnSODa and Cu/ZnSODb activities in source leaves were significantly different between transgenic and the wild type control (P<0.05; two-way ANOVA), with activity of the iron SOD higher in GS transgenic leaves than in the wild type (43% increase) while the Cu/Zn SOD a and b activities decreased (38% and 46% decrease respectively). These results are in line with the transcript-level response. Taken together, SOD transcript and protein activity assays support the initial microarray observation that some Cu/ZnSOD and FeSOD members exhibited differential expression responses to GS transgenic manipulation under drought conditions.

### DISCUSSION

The Populus genome contains two CCS and 12 SOD genes, including all major groups of SODs (Cu/ZnSOD, MnSOD and FeSOD) conserved in plants [15]. Relative to Arabidopsis, the Populus CCS/SOD families are about twice as large, due to duplication in all but one gene (FSD3). This is in sharp contrast to the predominantly single-copy nature of the Arabidopsis CCS/SOD orthologs (except AtFSD1), even though Arabidopsis has experienced two rounds of recent (α and β) whole-genome duplication versus one (Salicoid duplication) in Populus [56]. The preferential duplicate retention of essentially the entire complement of SODs and CCSs in Populus may hint at their importance in the response of woody perennials to oxidative stress. While expression of some duplicates, e.g., PtCSD2s and PtMSDs, remained similar in the tissues examined, patterns of transcript distribution of the other SOD pairs appeared to have diverged. For example, transcript levels of PtCSD3.2 were more evenly distributed across tissues, whereas PtCSD3.1 exhibited a biased expression in green tissues. In many cases, transcript levels, rather than tissue distribution patterns per se, have diverged between duplicate genes, with one copy showing higher expression than the other. The most notable examples are PtCSD1s, PtCSD3s, PtCCSs, and PtFSD2s. In the case of the PtFSD2 pair, the poorly expressed copy (PtFSD2.2) is predicted to encode a truncated protein. This suggests that PtFSD2.2 might have undergone pseudogenization following duplication, and may no longer be functional. Together, our data provide evidence that gene duplication/retention and, in some cases, differential regulation of duplicates have both contributed to the expansion and transcriptional diversity of the Populus SOD/CCS families, especially under stress conditions.

Transcript levels were highest for the chloroplast-localized SOD isoforms, e.g., PtCSD2s, PtCCSs, and PtFSD2.1, and these isoforms were also the ones that differed the most between GS poplar and the wild type under drought (FIGS. 6 and 7). Interestingly, the PtCSD2/PtCCS and PtFSD2.1 genes showed opposite patterns in response to drought, with the PtCSD2/PtCCS groups strongly down-regulated, and PtFSD2.1 up-regulated in GS poplar relative to the wild type. Down-regulation of plastidic CSDs with concomitant up-regulation of plastidic FSDs has also been reported in a number of species grown under Cu-limiting conditions [57]-[59]. It was suggested that suppression of Cu/ZnSOD during Cu-deficiency allows allocation of the Cu cofactor to plastocyanin, a major Cu-containing protein in the stroma, in order to sustain photosynthesis [54]. In Arabidopsis, this model was further supported by coordinated down-regulation of AtCCS in response to Cu-limitation [54]. Simultaneous induction of plastidic FeSOD is thought to protect chloroplasts against oxidative damage [57], as has been frequently reported in plants [60], [61]. In the case of GS poplars, net photosynthetic rates and chlorophyll contents were higher relative to the wild type, both before and during drought [7], [11]. This is consistent with an increased demand of Cu cofactor for photosynthetic electron transfer, and may occur at the expense of Cu/ZnSOD expression and protein accumulation, as observed in GS poplars. Thus, our results suggest that the Cu-modulated compensatory regulation between chloroplastic Cu/ZnSOD and FeSOD may be a common response to oxidative stress or transgenic manipulations that affect the photosynthesis. The cytosolic CSD1 and plastidic CSD2 and CCS are known to be regulated by microRNA 398 (miR398) [23], [49]. Although miRNAs were not investigated in the present study, stimulation of poplar miR398s by drought may be expected based on the strong down-regulation of their predicted targets, PtCSD1s, PtCSD2s and PtCCSs [62], [63], as has been reported for Medicago [64]. Another important yet relatively less emphasized role of miR398 is its involvement in the regulation of Cu homeostasis [65]. miRNA398 itself is negatively regulated by Cu, and its predicted targets, CDS1, CDS2, CCS and COX5b (mitochondrial cytochrome c oxidase subunit 5b) are Cu-containing proteins [49], [65]. Because metal homeostasis is closely coupled to cellular redox status and antioxidant defense, Yamasaki et al. [65] proposed that miR398 may be involved in the regulation of copper homeostasis.

The above analysis suggests that enhanced drought resistance of the GS poplars may involve altered Cu homeostasis and miRNA regulation. In addition to the miR398 targets (PtCSD1s, PtCSD2s and PtCCSs), several chloroplast-localized polyphenol oxidases (PPOs), another major Cu protein family in poplar [66], were down-regulated in GS poplars (FIG. 8). Populus PPOs were recently shown to be Cu-regulated by a new Cu-responsive miRNA, miR1444 [66]. The concept of coordinated down-regulation of major Cu proteins (CSD1, CSD2, CCS and PPO) by Cu-responsive miR398 and miR1444 is consistent with the Cu cofactor economy model in which Cu is diverted to plastocyanins, thus sustaining the increased photosynthetic rates observed in GS poplars [11]. Interestingly, miR398 was also found to be regulated by nutrient deficiencies, including N [67]. Taken together, our results suggest that, as a result of altered N metabolism and enhanced photosynthesis, drought tolerance in the GS poplars involves Cu- and miRNA-mediated antioxidant regulation.

SOD expression has also been reported to be regulated by ethylene. Kurepa et al. showed that ACC treatment of tobacco leaves increased transcript levels of an iron SOD and decreased transcript levels of a copper SOD [19]. GS poplars show higher levels of glutamine and glutamate, as well as γ-amino butyric acid (GABA) ([9] and data not shown). GABA is a non-proteinogenic amino acid often induced under biotic and abiotic stress conditions [68]. Kathiresan et al. reported that GABA stimulates ethylene biosynthesis in sunflower leaves [69]. Furthermore, glutamate decarboxylase, the principle enzyme in GABA biosynthesis, and ACC synthase and ACC oxidase show highly correlated expression patterns in pine [70]. Transcription of jasmonate-related genes is also affected by ectopic expression of GS in poplar tissues (manuscript in preparation). Thus, the present study shows that enhanced drought tolerance observed in GS poplars is accompanied by differential SOD gene expression patterns (i.e. higher iron SOD and lower Cu/Zn SOD expression) and suggests a relationship between GS expression and altered hormone homeostasis and GABA metabolism.

### CONCLUSIONS

The SOD/CCS families are significantly expanded in Populus relative to Arabidopsis, although both species have experienced independent rounds of whole genome duplication since they last shared a common ancestor. All but one of the SOD/CCS genes retained duplicated copies following whole genome duplication in Populus, while only one such pair was retained in Arabidopsis. Expression analysis revealed that some of the Populus paralogs have already diverged in their transcript abundance, tissue distribution patterns and/or stress response. We observed a coordinated down-regulation of the plastidic PtCDS2s and up-regulation of the plastidic PtFSDs, at the mRNA as well as activity levels, in drought-stressed GS transgenics. This is consistent with preferential allocation of Cu cofactor to plastocyanin to sustain high rates of photosynthesis in the GS transgenics under drought as previously reported. The model is further supported by down-regulation of several chloroplastidic PPOs, another major Cu protein, in the GS poplar during drought conditions. Our results suggest that alterations in N metabolism in GS transgenics cause differential regulation of genes involved in ROS protection under drought conditions leading to drought tolerance observed in the transgenics. Cu homeostasis and antioxidant regulation in response to altered N metabolism in the GS poplars need to be further investigated.

SUPPORTING TABLE 1
Proposed poplar SOD gene nomenclature based on SODs described for
Arabidopsis thallaina [15] and forward and reverse primers used for RT-qPCR analysis.
SEQ ID NOs are provided in parentheses.

| Populas | | | |
|---|---|---|---|
| trichocarpa | | | |
| SOD gene | | | |
| nomenclature | Locus | Forward (5′→3′) | Reverse (5′→3′) |
| PtCCS1 | POFTR_0001s08330 | GCCAGAAGTGCAGGAGTTG(1) | CAGTGAACAGAGTAAAACAAACACAGAG(11) |
| PtCCS2 | POFTR_0003s11830 | GCCAGAAGTGCAGGAGTTGG(1) | CAATGGCTGAACATGGTGC(12) |
| PtCSD1.1 | POFTR_0005s04590 | CTGTTGGTGATGATGGCACT(2) | ACTATGGCGGTGCTGTGG(13) |
| PtCSD1.2 | POFTR_0013s03160 | CTGTTGGTGATGATGGCACT(2) | GGCTTTCATATTTTTATTCAGAATCTATC(14) |
| PtCSD2.1 | POFTR_0002s01050 | ACTGGGAATGCAGGTGGA(3) | CCTGATAGTATTACTTTACACACTGAGAA(15) |
| PtCSD2.2a | POFTR_0011s01280 | CACTCACTCCTCCAAATCCA(4) | CACATCCCAAAATTAACATTAACATTA(16) |
| PtCSD2.2b | POFTR_0006s22520 | as for PtCSD2.2a | as for PtCSD2.2a |
| PtCSD3.1 | POFTR_0012s05140 | AGACAACTGGGAATGCAGGT(5) | CAGTTTCGAACAAGTATATTGGATC(17) |
| PtCSD3.2 | POFTR_0013s05350 | AGACAACTGGGAATGCAGGT(5) | GCACAAGTGTGTTGGACGAG(18) |
| PtMSD1 | POFTR_0012s08540 | CAAGCACACCTGCTCTGCA(6) | TTTCCATAGTTTCGATACACCAGTAA(19) |
| PtMSD2 | POFTR_0013s09270 | GGTGAAGTTTATGACAAAGAAAGC(7) | TAACATCCAACGAACCACGG(20) |
| PtFSD2.1 | POFTR_0015s12190 | TGGTGTCATGGGATGCAG(8) | AAGACAACGAAGGACGTGACA(21) |
| PtFSD2.2 | POFTR_0012s11400 | CACATCAACTTCCATGGAGAA(9) | CCGATGCCTGGATATTCATG(22) |
| PtFSD3 | POFTR_0005s09190 | GCAGAGGCATTCGTGAATCT(10) | CCAACATGACTGCATTTCTACC(23) |
| Arabidopsis | | | |
| thaliana SOD | | | |
| gene | | | |
| nomenclature | Locus | | |
| AtCCS | AT1G12520 | | |
| AtCDS1 | AT1G08830 | | |
| AtCDS2 | AT2G28190 | | |
| AtCDS3 | AT3G18100 | | |
| AtMSD | AT3G10920 | | |
| AtFSD1 | AT4G25100 | | |
| AtFSD2 | AT5G51100 | | |
| AtFSD3 | AT5G23310 | | |

SUPPORTING TABLE 2
Two-way ANOVA of observed transcript levels of SOD genes (all
tissues vs. water availability) in wild type plants. Genes are
sorted by P-values. Genes with P-values ≦0.05 appear in bold.

| | |
|---|---|
| Well-watered vs. Drought | Well-watered vs. Recovery |

| gene | P-value | gene | P-value |
|---|---|---|---|
| PtFSD2.1 | 3.65091E−06 | PtCCS1 | 1.20013E−05 |
| PtCSD1.1 | 0.001912709 | PtCSD3.2 | 0.000202235 |
| PtCSD3.2 | 0.006035689 | PtMSD1.1 | 0.001477703 |
| PtCSD2.2 | 0.006964789 | PtFSD2.2 | 0.013692673 |
| PtCCS1 | 0.007220917 | PtCSD2.2 | 0.025642525 |
| PtMSD1.1 | 0.013302107 | PtMSD1.2 | 0.042785742 |
| PtMSD1.2 | 0.013794331 | PtCSD3.1 | 0.053872381 |
| PtCSD1.2 | 0.028987756 | PtCSD2.1 | 0.056012715 |
| PtCCS2 | 0.029695719 | PtFSD3 | 0.071263661 |
| PtCSD3.1 | 0.042395926 | PtFSD2.1 | 0.324155133 |
| PtFSD3 | 0.069425621 | PtCCS2 | 0.419061163 |
| PtCSD2.1 | 0.704250451 | PtCSD1.2 | 0.644898431 |
| PtFSD2.2 | 0.742713244 | PtCSD1.1 | 0.751614377 |

### REFERENCES

1. Lower S S, Orians C M (2003) Soil nutrients and water availability interact to influence willow growth and chemistry but not leaf beetle performance. Entomologia Experimentalis et Applicata 107: 69-79
2. Quaye A, Laryea K, Mickson-Abeney S (2009) Soil Water and Nitrogen Interaction Effects on Maize (Zea mays L.) Grown on a Vertisol. Forestry, Horticulture, and Soil Science 3: 1-11.
3. Solomon S, Qin D, Manning M, Marquis M, Averyt K, et al. (2007) Climate change 2007: the physical science basis. Contribution of Working Group I to the Fourth Assessment Report of the Intergovermental Panel on Climate Change. New York: Cambridge University Press.
4. Marron N, Maury S, Rinaldi C, Brignolas F (2006) Impact of drought and leaf development stage on enzymatic antioxidant system of two Populus deltoides×nigra clones. Annals of Forest Science 63: 323-327
5. Lei Y, Yin C, Li C (2006) Differences in some morphological, physiological, and biochemical responses to drought stress in two contrasting populations of Populus przewalskii. Physiologia Plantarum 127: 182-191.
6. Cruz de Carvalho M (2008) Drought stress and reactive oxygen species. Plant signaling & behavior 3: 156-165.
7. Gallardo F, Fu J, Canton F, Garcia-Gutierrez A, Canovas F, et al. (1999) Expression of a conifer glutamine synthetase gene in transgenic poplar. Planta 210: 19-26
8. Jing Z P, Gallardo F, Pascual M B, Sampalo R, Romero J, et al. (2004) Improved growth in a field trial of transgenic hybrid poplar overexpressing glutamine synthetase. New Phytologist 164: 137-145.
9. Man H-M, Boriel R, El-Khatib R, Kirby E G (2005) Characterization of transgenic poplar with ectopic expression of pine cytosolic glutamine synthetase under conditions of varying nitrogen availability. The New phytologist 167: 31-39.
10. Coleman H D, Cánovas F M, Man H, Kirby E G, Mansfield S D (2012) Enhanced expression of glutamine synthetase (GS 1 a) confers altered fibre and wood chemistry in field grown hybrid poplar (Populus tremula×alba) (717-1B4). Plant biotechnology journal in press: 1-7.
11. el-Khatib R T, Hamerlynck E P, Gallardo F, Kirby E G (2004) Transgenic poplar characterized by ectopic expression of a pine cytosolic glutamine synthetase gene exhibits enhanced tolerance to water stress. Tree physiology 24: 729-736
12. Alscher R G, Erturk N, Heath L S (2002) Role of superoxide dismutases (SODs) in controlling oxidative stress in plants. Journal of experimental botany 53: 1331-1341
13. McCord J, Fridovich I (1969) Superoxide dismutase: an enzymic function for erythrocuprein (hemocuprein). J Biol Chem 244: 6049-6055.
14. Gill S S, Tuteja N (2010) Reactive oxygen species and antioxidant machinery in abiotic stress tolerance in crop plants. Plant physiology and biochemistry: PPB/Société franøaise de physiologie végétale 48: 909-930.
15. Kliebenstein D J, Monde R A, Last R L (1998) Superoxide dismutase in Arabidopsis: an eclectic enzyme family with disparate regulation and protein localization. Plant physiology 118: 637-650
16. Sandalio L M, Del Rio L A (1988) Intraorganellar distribution of superoxide dismutase in plant peroxisomes (glyoxysomes and leaf peroxisomes). Plant physiology 88: 1215-1218
17. Pradedova E V, Isheeva O D, Salyaev R K (2009) Superoxide dismutase of plant cell vacuoles. Biochemistry (Moscow) Supplement Series A: Membrane and Cell Biology 3: 24-32.
18. Srivastava V, Srivastava M K, Chibani K, Nilsson R, Rouhier N, et al. (2009) Alternative splicing studies of the reactive oxygen species gene network in Populus reveal two isoforms of high-isoelectric-point superoxide dismutase. Plant physiology 149: 1848-1859.
19. Kurepa J, Hérouart D, Van Montagu M, Inzé D (1997) Differential Expression of CuZn- and Fe-Superoxide Dismutase Genes of Tobacco during Development, Oxidative Stress, and Hormonal Treatments. Plant Cell Physiol 38: 463-470.
20. Bowler C, Alliotte T, Loose M De, Montagu M Van, Inze D (1989) The induction of manganese superoxide dismutase in response to stress in Nicotiana plumbaginifolia. The EMBO journal 8: 31-38.
21. Wu G, Wilen R, Robertson A J, Gusta L V (1999) Isolation, Chromosomal Localization, and Differential Expression of Mitochondrial Manganese Superoxide Dismutase and Chloroplastic Copper/Zinc Superoxide Dismutase Genes in Wheat. Plant physiology 120: 513-520.
22. Feng W, Hongbin W, Bing L, Jinfa W (2006) Cloning and characterization of a novel splicing isoform of the iron-superoxide dismutase gene in rice (Oryza sativa L.). Plant cell reports 24: 734-742.
23. Sunkar R, Kapoor A, Zhu J (2006) Posttranscriptional Induction of Two Cu/Zn Superoxide Dismutase Genes in Arabidopsis Is Mediated by Downregulation of miR398 and Important for Oxidative Stress Tolerance. The Plant Cell 18: 2051-2065
24. Dugas D V, Bartel B (2008) Sucrose induction of Arabidopsis miR398 represses two Cu/Zn superoxide dismutases. Plant molecular biology 67: 403-417.
25. Kim M D, Kim Y-H, Kwon S-Y, Yun D-J, Kwak S-S, et al. (2010) Enhanced tolerance to methyl viologen-induced oxidative stress and high temperature in transgenic potato plants overexpressing the CuZnSOD, APX and NDPK2 genes. Physiologia plantarum 140: 153-162.
26. Wang F-Z, Wang Q-B, Kwon S-Y, Kwak S-S, Su W-A (2005) Enhanced drought tolerance of transgenic rice plants expressing a pea manganese superoxide dismutase. Journal of Plant Physiology 162: 465-472.
27. Wang Y C, Qu G Z, Li H Y, Wu Y J, Wang C, et al. (2010) Enhanced salt tolerance of transgenic poplar plants expressing a manganese superoxide dismutase from Tamarix androssowii. Molecular biology reports 37: 1119-1124.
28. Altschul S F, Madden T L, Schaffer A A, Zhang J, Zhang Z, et al. (1997) Gapped BLAST and PSI-BLAST: a new generation of protein database search programs. Nucleic acids research 25: 3389-3402
29. Wilkins M R, Gasteiger E, Bairoch A, Sanchez J C, Williams K L, et al. (1999) Protein identification and analysis tools in the ExPASy server. Methods in molecular biology (Clifton, N.J.) 112: 531-552
30. Larkin M, Blackshields G, Brown N P, Chenna R, McGettigan P, et al. (2007) Clustal W and Clustal X version 2.0. Bioinformatics (Oxford, England) 23: 2947-2948.
31. Tamura K, Peterson D, Peterson N, Stecher G, Nei M, et al. (2011) MEGA5: molecular evolutionary genetics analysis using maximum likelihood, evolutionary distance, and maximum parsimony methods. Molecular biology and evolution 28: 2731-2739.
32. Emanuelsson O, Brunak S, Von Heijne G, Nielsen H (2007) Locating proteins in the cell using TargetP, SignalP and related tools. Nature protocols 2: 953-971.
33. Petersen T N, Brunak S, Von Heijne G, Nielsen H (2011) SignalP 4.0: discriminating signal peptides from transmembrane regions. Nature methods 8: 785-786.
34. Emanuelsson O, Nielsen H, Von Heijne G (1999) ChloroP, a neural network-based method for predicting chloroplast transit peptides and their cleavage sites. Protein science: a publication of the Protein Society 8: 978-984
35. Claros M G, Vincens P (1996) Computational method to predict mitochondrially imported proteins and their targeting sequences. European journal of biochemistry/FEBS 241: 779-786
36. Neuberger G, Maurer-Stroh S, Eisenhaber B, Hartig A, Eisenhaber F (2003) Prediction of Peroxisomal Targeting Signal 1 Containing Proteins from Amino Acid Sequence. Journal of Molecular Biology 328: 581-592.
37. Liao Z, Chen M, Guo L, Gong Y, Tang F, et al. (2004) Rapid Isolation of High-Quality Total RNA from Taxus and Ginkgo. Preparative Biochemistry and Biotechnology 34: 209-214.
38. Untergasser A, Nijveen H, Rao X, Bisseling T, Geurts R, et al. (2007) Primer3Plus, an enhanced web interface to Primer3. Nucleic acids research 35: W71-4.
39. Brunner A M, Yakovlev I, Strauss S H (2004) Validating internal controls for quantitative plant gene expression studies. BMC plant biology 4: 14.
40. Vandesompele J, De Preter K, Pattyn F, Poppe B, Van Roy N, et al. (2002) Accurate normalization of real-time quantitative RT-PCR data by geometric averaging of multiple internal control genes. Genome biology 3:
41. Ruijter J M, Ramakers C, Hoogaars W M H, Karlen Y, Bakker O, et al. (2009) Amplification efficiency: linking baseline and bias in the analysis of quantitative PCR data. Nucleic acids research 37: e45.
42. De Hoon M J L, Imoto S, Miyano S (2002) Statistical analysis of a small set of time-ordered gene expression data using linear splines. Bioinformatics 18: 1477-1485.
43. Saldanha A J (2004) Java Treeview—extensible visualization of microarray data. Bioinformatics (Oxford, England) 20: 3246-3248.
44. Bradford M M (1976) A rapid and sensitive method for the quantitation of microgram quantities of protein utilizing the principle of protein-dye binding. Anal Biochem 72: 248-254.
45. Weydert C J, Cullen J J (2010) Measurement of superoxide dismutase, catalase and glutathione peroxidase in cultured cells and tissue. Nature protocols 5: 51-66.
46. Fridovich I (1975) Superoxide dismutases. Annu Rev Biochem 44: 147-159.
47. Abràmoff M D, Magalhãles P J, Ram S J (2004) Image Processing with ImageJ. Biophotonics international 11: 36-42.
48. Tuskan G a, Difazio S, Jansson S, Bohlmann J, Grigoriev I, et al. (2006) The genome of black cottonwood, Populus trichocarpa (Torr. & Gray). Science (New York, N.Y.) 313: 1596-1604.
49. Cohu C M, Abdel-Ghany S E, Gogolin Reynolds K a, Onofrio A M, Bodecker J R, et al. (2009) Copper delivery by the copper chaperone for chloroplast and cytosolic copper/zinc-superoxide dismutases: regulation and unexpected phenotypes in an Arabidopsis mutant. Molecular plant 2: 1336-1350.
50. Jiang K, Goertzen L R (2011) Spliceosomal intron size expansion in domesticated grapevine (Vitis vinifera). BMC research notes 4: 52.
51. Pufahl R (1997) Metal Ion Chaperone Function of the Soluble Cu(I) Receptor Atx1. Science 278: 853-856.
52. Bordo D, Djinovic K, Bolognesi M (1994) Conserved patterns in the Cu,Zn superoxide dismutase family. Journal of Molecular Biology 238: 366-386.
53. Van Camp W, Bowler C, Villarroel R, Tsang E W, Van Montagu M, et al. (1990) Characterization of iron superoxide dismutase cDNAs from plants obtained by genetic complementation in Escherichia coli. Proceedings of the National Academy of Sciences of the United States of America 87: 9903-9907
54. Abdel-Ghany S E, Burkhead J L, Gogolin K a, Andrés-Colás N, Bodecker J R, et al. (2005) AtCCS is a functional homolog of the yeast copper chaperone Ccs1/Lys7. FEBS letters 579: 2307-2312.
55. Wintz H, Vulpe C (2002) Plant copper chaperones. Biochemical Society transactions 30: 732-735.
56. Jiao Y, Wickett N J, Ayyampalayam S, Chanderbali A S, Landherr L, et al. (2011) Ancestral polyploidy in seed plants and angiosperms. Nature 473: 97-100.
57. Abdel-Ghany S E, Müller-Moulé P, Niyogi K K, Pilon M, Shikanai T (2005) Two P-Type ATPases Are Required for Copper Delivery in Arabidopsis thaliana Chloroplasts. The Plant Cell 17: 1233-1251
58. Tewari R K, Kumar P, Sharma P N (2006) Antioxidant responses to enhanced generation of superoxide anion radical and hydrogen peroxide in the copper-stressed mulberry plants. Planta 223: 1145-1153.
59. Cohu C M, Pilon M (2007) Regulation of superoxide dismutase expression by copper availability. Physiologia Plantarum 129: 747-755.
60. Tsang E W, Bowler C, Hérouart D, Van Camp W, Villarroel R, et al. (1991) Differential regulation of superoxide dismutases in plants exposed to environmental stress. The Plant cell 3: 783-792
61. Myouga F, Hosoda C, Umezawa T, lizumi H, Kuromori T, et al. (2008) A heterocomplex of iron superoxide dismutases defends chloroplast nucleoids against oxidative stress and is essential for chloroplast development in Arabidopsis. The Plant cell 20: 3148-3162.
62. Jia X, Wang W-X, Ren L, Chen Q-J, Mendu V, et al. (2009) Differential and dynamic regulation of miR398 in response to ABA and salt stress in Populus tremula and Arabidopsis thaliana. Plant molecular biology 71: 51-59.
63. Lu Y, Feng Z, Bian L, Xie H, Liang J (2011) miR398 regulation in rice of the responses to abiotic and biotic stresses depends on CSD1 and CSD2 expression. Functional Plant Biology 38: 44-53.
64. Trindade I, Capitaõ C, Dalmay T, Fevereiro M P, Santos D M Dos (2010) miR398 and miR408 are up-regulated in response to water deficit in Medicago truncatula. Planta 231: 705-716.
65. Yamasaki H, Abdel-Ghany S E, Cohu C M, Kobayashi Y, Shikanai T, et al. (2007) Regulation of copper homeostasis by micro-RNA in Arabidopsis. The Journal of biological chemistry 282: 16369-16378.
66. Ravet K, Danford F L, Dihle A, Pittarello M, Pilon M (2011) Spatiotemporal analysis of copper homeostasis in Populus trichocarpa reveals an integrated molecular remodeling for a preferential allocation of copper to plastocyanin in the chloroplasts of developing leaves. Plant physiology 157: 1300-1312.
67. Hsieh L-C, Lin S-I, Shih A C-C, Chen J-W, Lin W-Y, et al. (2009) Uncovering small RNA-mediated responses to phosphate deficiency in Arabidopsis by deep sequencing. Plant physiology 151: 2120-2132.
68. Roberts M R (2007) Does GABA Act as a Signal in Plants? Plant signaling & behavior 2: 408-409.
69. Kathiresan A, Tung P, Chinnappa C, Reid D (1997) y-Aminobutyric Acid Stimulates Ethylene Biosynthesis in Sunflower. Plant physiology 115: 129-135.
70. Molina-Rueda J J, Pascual M B, Cánovas F M, Gallardo F (2010) Characterization and developmental expression of a glutamate decarboxylase from maritime pine. Planta 232: 1471-1483.
71. Man H, Pollmann S, Weiler E W, Kirby E G (2011) Increased glutamine in leaves of poplar transgenic with pine GS 1 a caused greater anthranilate synthetase α-subunit (ASA1) transcript and protein abundances: an auxin-related mechanism for enhanced growth in GS transgenics? Journal of experimental botany 62: 4423-4431.
72. Parker M W, Blake C C (1988) Iron- and manganese-containing superoxide dismutases can be distinguished by analysis of their primary structures. FEBS letters 229: 377-382
73. Yamakura F (1984) Destruction of tryptophan residues by hydrogen peroxide in iron-superoxide dismutase. Biochemical and biophysical research communications 122: 635-641.
74. Wang H-Q, Tuominen L K, Tsai C-J (2011) SLIM: a sliding linear model for estimating the proportion of true null hypotheses in datasets with dependence structures. Bioinformatics (Oxford, England) 27: 225-231

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A plant expression cassette, which comprises a nucleic acid sequence selected from the group consisting of PtFSD2.1, PtFSD3 and PtYSL operably linked to a promoter.

2. The expression cassette of claim 1, wherein the sequences are from a gymnosperm.

3. The expression cassette of claim 2, wherein the sequence is from the genus Pinus.

4. The expression cassette of claim 3, wherein the sequence is from Pinus sylvestris.

5. The expression cassette of claim 2, in which the promoter is the cauliflower mosaic virus 35S promoter.

6. The expression cassette of claim 5, which further comprises the NOS terminator sequence operably linked to the glutamine synthetase coding sequence.

7. A vector, comprising the expression cassette of claim 1.

8. The vector of claim 7, which is an Agrobacterium binary vector.

9. The vector of claim 8, wherein the vector is pBIN19.

10. The vector of claim 9, which further comprises the neomycin phosphotransferase II coding sequence.

11. A method of producing a plant with improved resistance to drought stress by transforming in vitro said plant with the expression cassette of claim 1.

12. The method of claim 11, wherein the plant is a woody perennial.

13. The method of claim 12, wherein the plant is in the family Salicaceae.

14. The method of claim 11, wherein the plant is in the genus Populus.

15. The method of claim 14, wherein the plant is the hybrid Populus tremula×P. alba.

16. The method of claim 15, wherein the plant is clone INRA 717 1-B4 of hybrid Populus tremula×P. alba.

17. The method of claim 11, wherein the transformation step uses the Agrobacterium tumifaciens method.

18. The method of claim 12, wherein the transformation step further uses the vector of claim 9.

19. A transgenic plant produced by the method of claim 11.

20. A reproductive unit from the transgenic plant of claim 14.

21. A cell from the transgenic plant of claim 20.

22. A transgenic plant with an improved resistance to drought stress which is a woody perennial and comprises at least one transgene that comprises the coding sequence of at least one gene selected from the group consisting of PtFSD2.1, PtFSD3 and PtYSL.

23. The transgenic plant of claim 22, wherein the gene is from a gymnosperm.

24. The transgenic plant of claim 22, wherein the at least one gene is from Pinus sylvestris.

25. A panel of isolated drought resistance plant biomarkers, said biomarkers being nucleic acids encoding part or all of genes listed in Table 1, said nucleic acids being operably linked to solid support or detectably labeled.
